(19) United States
(12) Patent Application Publication
(10) Pub. No.: US 2007/0185099 A1
(43) Pub. Date: Aug. 9, 2007

(54) Triazolopyrimidine compounds and their use for controlling pathogenic fungi

(76) Inventors: Carsten Blettner, Hong Kong (CN); Frank Schieweck, Hessheim (DE); Jordi Tormi i Blasco, Laudenbach (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Anja Schwogler, Mannheim (DE); Oliver Wagner, Neustadt (DE); John-Bryan Speakman, Bobenheim (DE); Thorsten Jabs, Hassloch (DE); Siegfried Strathmann, Limburgerhof (DE); Ulrich Schofl, Bruhl (DE); Maria Scherer, Godramstein (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 11/628,852

(22) PCT Filed: Jun. 24, 2005
(86) PCT No.: PCT/EP05/06855

§ 371 Date: Dec. 8, 2006

(30) Foreign Application Priority Data
Jun. 25, 2004 (DE) 10 2004 030 816.0

(51) Int. Cl. A01N 43/90; C07D 487/04

(52) U.S. Cl. 514/227.8; 514/259.31; 544/263; 514/234.5; 514/252.16; 544/60; 544/120

(57) ABSTRACT

The present invention relates to novel triazolopyrimidine compounds of the formula I 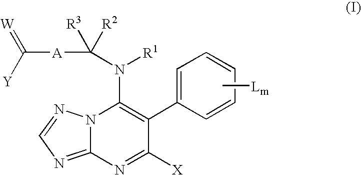 in which:
X is halogen, cyano, C₁-C₄-alkyl, C₁-C₄-haloalkyl, C₁-C₄-alkoxy or C₁-C₂-haloalkoxy; W is oxygen or sulfur;
Y is O—R⁴ or a group NR⁵R⁶;
A is a chemical bond or a group CR⁷R⁸;
   and the variables L, R¹ to R⁷ are as defined in claim 1. The present invention furthermore provides the use of the triazolopyrimidine compounds of the formula I, their tautomers and their agriculturally acceptable salts for controlling phytopathogenic fungi (=harmful fungi) and a method for controlling phytopathogenic harmful fungi, which method comprises treating the fungi or the materials, plants, the soil or seed to be protected against fungal attack with an effective amount of a compound of the formula I, a tautomer of I and/or an agriculturally acceptable salt of I or a tautomer thereof.

The present invention relates to novel triazolopyrimidine compounds and to their use for controlling harmful fungi and also to crop protection compositions comprising such compounds as active ingredient.

EP-A 71792, U.S. Pat. No. 5,994,360, EP-A 550113, WO-A 94/20501, EP-A 834 513, WO-A 98/46608 and WO 03/080615 describe fungicidally active triazolo[1,5a]pyrimidines which carry an optionally substituted phenyl group in the 6-position of the azolopyrimidine ring and NH₂ or a primary or secondary amino group in the 7-position.

With respect to their fungicidal action, some of the triazolopyrimidines known from the prior art and having an amino group in the 7-position are not entirely satisfactory, or they have unwanted properties, such as poor compatibility with useful plants.

Accordingly, it is an object of the present invention to provide novel compounds having better fungicidal activity and/or better compatibility with useful plants.

Surprisingly, this object is achieved by triazolopyrimidine compounds of the formula I 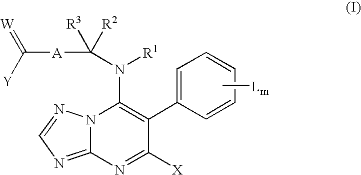
in which:
X is halogen, cyano, C₁-C₄-alkyl, C₁-C₄-haloalkyl, C₁-C₄-alkoxy or C₁-C₂-haloalkoxy;
W is oxygen or sulfur;
Y is O—R⁴ or a group NR⁵R⁶;
A is a chemical bond or a group CR⁷R⁸;
the radicals L independently of one another are halogen, C₁-C₆-alkyl, C₂-C₆-alkenyl, C₁-C₆-haloalkyl, C₁-C₆-alkoxy, nitro, amino, NHR, NR₂, cyano, S(═O)ₙA¹ or C(═O)A², in which
   R independently of one another are C₁-C₈-alkyl or C₁-C₈-alkylcarbonyl;
   A¹ is hydrogen, hydroxyl, C₁-C₈-alkyl, NH₂, C₁-C₈-alkylamino or di(C₁-C₈-alkyl)amino;
   n is 0, 1 or 2;
   A² is C₂-C₈-alkenyl, C₁-C₈-alkoxy, C₁-C₆-haloalkoxy, hydrogen, hydroxyl, C₁-C₈-alkyl, NH₂, C₁-C₈-alkylamino or di-(C₁-C₈-alkyl)amino;
m is 0 or 1, 2, 3, 4 or 5;
R¹ is hydrogen, C₁-C₄-alkyl, formyl, C₁-C₄-alkylcarbonyl or C₁-C₄-alkoxycarbonyl or together with R² is C₃-C₆-alkylene where 1 carbon atom may be replaced by an oxygen atom or a sulfur atom and which may optionally carry 1, 2, 3 or 4 C₁-C₄-alkyl groups and/or 1 or 2 radicals R^{a};
   R^{a}: is halogen, OH, C₁-C₄-alkoxy or C₁-C₄-alkoxycarbonyl;
R² is hydrogen, C₁-C₆-alkyl which may have a radical R^{b}, C₁-C₄-haloalkyl, C₁-C₄-alkoxy, C₃-C₆-cycloalkyl or phenyl which optionally carries 1, 2 or 3 radicals R^{c};
   R^{b}: is OR⁹, SR¹⁰, NR¹¹R¹², COOR¹³, CONR¹⁴R¹⁵, NHC(═NR¹⁶)NR¹⁴R¹⁵, phenyl which optionally carries 1, 2 or 3 radicals R^{c}, 5- or 6-membered heteroaryl which has 1 nitrogen atom and optionally 1 or 2 further heteroatoms selected from the group consisting of O, S and N as ring members and which optionally carries 1 or 2 radicals R^{b} or can have a fused-on phenyl ring which for its part can have 1 or 2 radicals R^{b}, or is C₃-C₆-cycloalkyl;
   R^{c}: is halogen, C₁-C₄-alkyl, C₁-C₄-haloalkyl, OH, C₁-C₄-alkoxy or C₁-C₄-alkoxycarbonyl;
R³ is hydrogen, C₁-C₄-alkyl or C₁-C₄-alkoxy or together with R² is C₂-C₆-alkylene where 1 carbon atom may be replaced by an oxygen atom or a sulfur atom and which may optionally carry 1, 2, 3 or 4 C₁-C₄-alkyl groups and/or 1 or 2 radicals R^{a};
R⁴ is hydrogen, C₁-C₈-alkyl, hydroxy-C₁-C₄-alkyl, C₁-C₄-alkoxy-C₁-C₄-alkyl, hydroxy-C₁-C₄-alkoxy-C₁-C₄-alkyl, C₁-C₈-haloalkyl, C₂-C₈-alkenyl, C₂-C₈-haloalkenyl, C₃-C₆-cycloalkyl, C₃-C₆-cycloalkyl-C₁-C₄-alkyl, C₃-C₆-halocycloalkyl, C₃-C₈-cycloalkenyl, phenyl, phenyl-C₁-C₄-alkyl, where phenyl in the two last-mentioned radicals may have 1, 2 or 3 of the substituents R^{d} below:
   R^{d}: is halogen, cyano, nitro, hydroxyl, mercapto, amino, carboxyl, aminocarbonyl, aminothiocarbonyl, C₁-C₄-alkyl, C₁-C₄-haloalkyl, C₁-C₄-alkoxy, C₁-C₄-haloalkoxy, C₁-C₄-alkylthio, C₁-C₄-alkylamino, C₁-C₄-dialkylamino, C₁-C₄-alkylcarbonyl, C₁-C₄-alkylsulfonyl, C₁-C₄-alkylsulfinyl, C₁-C₄-alkoxycarbonyl, C₁-C₄-alkylcarbonyloxy, C₁-C₄-alkylaminocarbonyl, C₁-C₄-dialkylaminocarbonyl,
   or R⁴ together with one of the radicals R¹, R², R³ or R⁷ is C₂-C₆-alkylene where 1 carbon atom may be replaced by an oxygen atom or a sulfur atom and which may optionally carry 1, 2, 3 or 4 radicals selected from the group consisting of halogen and C₁-C₄-alkyl and/or 1 or 2 radicals R^{a};
R⁵, R⁶ independently of one another are hydrogen, C₁-C₈-alkyl, C₃-C₆-cycloalkyl, C₃-C₆-cycloalkyl-C₁-C₄-alkyl, or together with the nitrogen atom to which they are attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups;
   or one of the radicals R⁵ or R⁶ together with one of the radicals R¹, R², R³ or R⁷ is C₂-C₆-alkylene where 1 carbon atom may be replaced by an oxygen atom or a sulfur atom and which may optionally carry 1, 2, 3 or 4 radicals selected from the group consisting of halogen and C₁-C₄-alkyl and/or 1 or 2 radicals R^{a};
R⁷, R⁸ independently of one another are hydrogen, C₁-C₄-alkyl or C₁-C₄-alkoxy or one of the radicals R⁷ or R⁸ together with one of the radicals R¹ or R² is C₂-C₆-alkylene where 1 carbon atom may be replaced by an oxygen atom or a sulfur atom and which may optionally carry 1, 2, 3 or 4 C₁-C₄-alkyl groups and/or 1 or 2 radicals R^{a};
R⁹ is hydrogen, C₁-C₈-alkyl, formyl or C₁-C₈-alkylcarbonyl;
R¹⁰ is hydrogen or C₁-C₄-alkyl;
R¹¹, R¹² independently of one another are hydrogen, C₁-C₈-alkyl, C₃-C₆-cycloalkyl, C₃-C₆-cycloalkyl-C₁-C₄-alkyl, or together with the nitrogen atom to which they are attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups, where one of the radicals R¹¹, R¹² may also be formyl, C₁-C₈-alkylcarbonyl or C₁-C₈-alkylthiocarbonyl;
R¹³ is hydrogen, C₁-C₈-alkyl, hydroxy-C₁-C₄-alkyl, C₁-C₄-alkoxy-C₁-C₄-alkyl, hydroxy-C₁-C₄-alkoxy-C₁-C₄-alkyl, C₁-C₈-haloalkyl, C₂-C₈-alkenyl, C₂-C₈-haloalkenyl, C₃-C₆-cycloalkyl, C₃-C₆-cycloalkyl-C₁-C₄-alkyl, C₃-C₆-halocycloalkyl, C₃-C₈-cycloalkenyl, phenyl, phenyl-C₁-C₄-alkyl, where phenyl in the two last-mentioned radicals may have 1, 2 or 3 of the abovementioned substituents R^{d};
R¹⁴, R¹⁵ independently of one another are hydrogen, C₁-C₈-alkyl, C₃-C₆-cycloalkyl, C₃-C₆-cycloalkyl-C₁-C₄-alkyl, or together with the nitrogen atom to which are they attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups;
R¹⁶ is hydrogen or C₁-C₄-alkyl;
   and by the agriculturally acceptable salts of the compounds I.

The present invention therefore provides the triazolopyrimidine compounds of the formula I and their agriculturally acceptable salts.

The present invention furthermore provides the use of the triazolopyrimidine compounds of the formula I, their tautomers and their agriculturally acceptable salts for controlling phytopathogenic fungi (=harmful fungi), and also a method for controlling phytopathogenic fungi, which method comprises treating the fungi or the materials, plants, the soil or seed to be protected against fungal attack with an effective amount of a compound of the formula I, a tautomer of I and/or with an agriculturally acceptable salt of I or a tautomer thereof.

The present invention further provides compositions for controlling harmful fungi, which compositions comprise at least one compound of the formula I, a tautomer of I and/or an agriculturally acceptable salt thereof or a tautomer thereof and at least one liquid or solid carrier.

Depending on the substitution pattern, the compounds of the formula I and their tautomers may have one or more centers of chirality, in which case they are present as pure enantiomers or pure diastereomers or as enantiomer or diastereomer mixtures. The invention provides both the pure enantiomers or diastereomers and also their mixtures.

Suitable agriculturally useful salts are especially the salts of those cations or the acid addition salts of those acids whose cations and anions, respectively, have no adverse effect on the fungicidal action of the compounds I. Suitable cations are thus in particular the cations of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and also the anions of C₁-C₄-alkanoic acids, preferably formate, acetate, propionate and butyrate. They can be formed by reacting I with an acid of the corresponding anion, preferably of hydrochloric acid, hydrobromic acid, sulfuric acid, phosphoric acid or nitric acid.

In the definitions of the variables given in the formulae above, collective terms are used which are generally representative of the substituents in question. The term Cₙ-Cₘ denotes the number of carbon atoms possible in each case in the substituent or substituent moiety:

halogen: fluorine, chlorine, bromine and iodine;

alkyl and all alkyl moieties in alkoxy, alkylthio, alkoxyalkyl, alkoxyalkoxy, alkylamino and dialkylamino: saturated straight-chain or branched hydrocarbon radicals having 1 to 4, to 6, to 8 or to 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 4 or to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;

alkenyl: monounsaturated straight-chain or branched hydrocarbon radicals having 2 to 4, to 6, to 8 or to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkadienyl: diunsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;

alkynyl: straight-chain or branched hydrocarbon groups having 2 to 4, 2 to 6, 2 to 8 or 2 to 10 carbon atoms and a triple bond in any position, for example C₂-C₆-alkynyl, such as ethynyl, 1-propinyl, 2-propinyl, 1-butynyl, 2-butynyl, 3-butynyl, 1-methyl-2-propinyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propinyl, 1-ethyl-2-propinyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propinyl;

cycloalkyl: monocyclic saturated hydrocarbon groups having 3 to 8, preferably to 6, carbon ring members, such as cyclopropyl, cyclobutyl, cyclopentyl and cyclohexyl;

cycloalkenyl: monocyclic monounsaturated hydrocarbon groups having 3 to 8, preferably to 6, carbon ring members, such as cyclopenten-1-yl, cyclopenten-3-yl, cyclohexen-1-yl, cyclohexen-3-yl and cyclohexen-4-yl;

bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, Bicyclo[2.2.2]oct-2-yl, bicyclo[3.3.0]octyl and bicyclo[4.4.0]decyl;

alkylamino: an alkyl group attached via an NH group, in which alkyl is one of the alkyl radicals mentioned above having generally 1 to 6 and in particular 1 to 4 carbon atoms, such as methylamino, ethylamino, n-propylamino, isopropylamino, n-butylamino and the like;

dialkylamino: a radical of the formula N(alkyl)₂ in which alkyl is one of the alkyl radicals mentioned above having generally 1 to 6 and in particular 1 to 4 carbon atoms, for example dimethylamino, diethylamino, methylethylamino, N-methyl-N-propylamino and the like;

C₁-C₄-alkoxy: an alkyl group, attached via oxygen, having 1 to 4 carbon atoms: for example methoxy, ethoxy, n-propoxy, 1-methylethoxy, butoxy, 1-methylpropoxy, 2-methylpropoxy or 1,1-dimethylethoxy;

C₁-C₆-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

C₁-C₆-haloalkoxy: C₁-C₄-haloalkoxy as mentioned above, and also, for example, 5-fluoropentoxy, 5-chloropentoxy, 5-bromopentoxy, 5-iodpentoxy, undecafluoropentoxy, 6-fluorohexoxy, 6-chlorohexoxy, 6-bromohexoxy, 6-iodohexoxy or dodecafluorohexoxy;

alkenyloxy: alkenyl as mentioned above which is attached via an oxygen atom, for example C₂-C₆-alkenyloxy, such as vinyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

alkylene: a linear saturated hydrocarbon chain having 2 to 6 and in particular 2 to 4 carbon atoms, such as ethane-1,2-diyl, propane-1,3-diyl, butane-1,4-diyl, pentane-1,5-diyl or hexane-1,6-diyl;

a five- or six-membered saturated or partially unsaturated heterocycle which contains one, two, three or four heteroatoms from the group consisting of oxygen, nitrogen and sulfur as ring members: for example mono- and bicyclic heterocycles (heterocyclyl) containing, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 1-piperidinyl, 2-piperidinyl, 3-piperidinyl, 4-piperidinyl, 4-morpholinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding-ylidene radicals;

a seven-membered saturated or partially unsaturated heterocycle which contains one, two, three or four heteroatoms from the group consisting of oxygen, nitrogen and sulfur as ring members: for example mono- and bicyclic heterocycles having 7 ring members which contain, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals;

a five- or six-membered aromatic heterocycle which contains one, two, three or four heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

With a view to the use of fungicides, preference is given to those compounds of the formula I in which the variables and X, A, W, R¹, R², R³, m and L independently of one another and in particular in combination are as defined below.
X is halogen, especially chlorine, or C₁-C₄-alkyl, especially methyl, particularly preferably halogen and very particularly preferably chlorine;
A is a chemical bond or CH₂, in particular a chemical bond;
W is oxygen;
R¹ is hydrogen or C₁-C₄-alkyl, or R¹ together with R² forms a linear or branched C₂-C₆-alkylene group, in particular a linear C₃-C₄-alkylene group;
R² is C₂-C₆-alkyl, trifluoromethyl or a group (CH₂)ₖ—R^{b} in which k is 1 or 2 and R^{b} is as defined above;
R³ is hydrogen;
m is 1, 2, 3 or 4, in particular 1, 2 or 3;
L is halogen, cyano, C₁-C₆-alkyl, C₁-C₆-haloalkyl, C₁-C₆-alkoxy or C₁-C₆-alkoxycarbonyl, in particular fluorine, bromine, cyano, C₁-C₄-alkyl, C₁-C₄-haloalkyl, C₁-C₄-alkoxy or C₁-C₄-alkoxycarbonyl, particularly preferably fluorine, chlorine, C₁-C₂-alkyl, such as methyl or ethyl, C₁-C₂-fluoroalkyl, such as trifluoromethyl, C₁-C₂-alkoxy, such as methoxy, or C₁-C₂-alkoxycarbonyl, such as methoxycarbonyl. Preference is furthermore given to compounds I in which at least one group L is located in the ortho-position to the point of attachment to the triazolopyrimidine skeleton.

In a preferred embodiment, Y is a group O—R⁴, where R⁴ is as defined above. Here, R⁴ is in particular C₁-C₄-alkyl, C₁-C₄-alkoxy-C₁-C₂-alkyl or C₃-C₄-alkenyl and in particular H, methyl, ethyl, n-propyl, 2-methoxyethyl, 2-ethoxyethyl or 2-propenyl (=allyl). In a further preferred embodiment, Y is a group O—R⁴ where R⁴, together with the radical R², is a C₂-C₄-alkylene group.

In another preferred embodiment, Y is a group N—R⁵R⁶ in which R⁵, R⁶ are as defined above. Here, R⁵ is in particular H, C₁-C₄-alkyl or C₃-C₄-alkenyl and in particular methyl, ethyl, n-propyl or n-propenyl. R⁶ is in particular H, C₁-C₄-alkyl or C₃-C₄-alkenyl and in particular H, methyl, ethyl or n-propyl. R⁵ and R⁶ together with the nitrogen atom to which they are attached may also form a saturated 5- to 7-membered nitrogen heterocycle which may optionally have a further heteroatom selected from the group consisting of O, S and N as ring member and which may optionally have 1 to 4 methyl groups: in this case, for example, Y is 1-pyrrolidinyl, 1-piperidinyl, 4-morpholinyl, 4-thiomorpolinyl or 4-methylpiperazin-1-yl. In a further preferred embodiment, Y is a group N—R⁵R⁶ in which R⁵, together with the radical R², is a C₂-C₄-alkylene group and R⁶ is hydrogen.

Among the compounds of the formula I, particularly preference is given to those in which the phenyl group substituted by Lₘ is the group of the formula 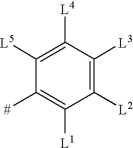
in which # is the point of attachment to the triazolopyrimidine skeleton and
L¹ is fluorine, chlorine, CH₃ or CF₃;
L², L⁴ independently of one another are hydrogen, chlorine or fluorine, where L⁴ may also be NO₂;
L³ is hydrogen, fluorine, chlorine, cyano, NO₂, C₁-C₄-alkyl, especially CH₃, C₁-C₄-alkoxy, especially OCH₃, or CO(A²), where A² is OH, C₁-C₄-alkoxy, especially OCH₃, NH₂, C₁-C₄-alkylamino; and
L⁵ is hydrogen, fluorine, chlorine or C₁-C₄-alkyl, especially CH₃.

Among the compounds I, particular preference is given to those in which R¹ is hydrogen or R¹ together with R² forms a linear or branched C₂-C₆-alkylene group, in particular a linear C₃-C₅-alkylene group. Here, R³ is in particular hydrogen.

Particular preference is also given to those compounds of the formula I in which R² is C₂-C₆-alkyl. Here, R³ is in particular hydrogen. In this case, R¹ is likewise in particular hydrogen.

Among these, particular preference is given to those compounds of the formula I in which R³ is hydrogen, W is oxygen and Y is a group OR⁴ which has the meanings mentioned above and in particular the preferred meanings, and the radicals R¹ and R² correspond to those of the following amino acids: proline, pipecolinic acid, leucine, isoleucine, methionine, phenylalanine, tyrosine and valine. In other words, the group of the formula 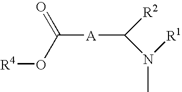
is derived from one of the α-amino acids mentioned above or an ester, in particular a C₁-C₄-alkyl ester or a C₃-C₄-alkenyl ester.

Another preferred embodiment of the invention relates to compounds I in which R² is a group (CH₂)ₖ—R^{b}, where k is 1 or 2 and R^{b} is as defined above. Here, R³ is in particular hydrogen. In this case, R¹ is likewise in particular hydrogen. Here, R^{b} has in particular the following meanings: phenyl, 4-hydroxyphenyl, 3,4-dihydroxyphenyl, imidazol-4-yl, indol-3-yl, 5-hydroxindol-3-yl, C₁-C₄-alkylthio, especially S—CH₃, C₁-C₄-alkoxy or C₁-C₄-alkoxycarbonyl.

If Y in formula I is a group NR⁵R⁶, R⁵ and R⁶ independently of one another have the following meanings: H or C₁-C₄-alkyl.

In the groups OR⁹, SR¹⁰, NR¹¹R¹², C(O)OR¹³, CONR¹⁴R¹⁵ and C(═N—R¹⁶)NR¹⁴R¹⁵, NHC(W)R⁶, C(W)R¹⁷ and NR¹⁸R¹⁹, the variables have in particular the meanings given below:

R⁹ is in particular H, C₁-C₄-alkyl, C(O)H or C₁-C₄-alkylcarbonyl;

R¹⁰ is in particular H or C₁-C₄-alkyl;

R¹¹ and R¹² are in particular H, C₁-C₄-alkyl, C₁-C₄-alkylcarbonyl or

C₁-C₄-alkyl(thiocarbonyl). NR¹¹R¹² is in particular NH₂, NHCH₃, NHC₂H₅, N(CH₃)₂, N(C₂H₅)CH₃, NHC(O)CH₃ or NHC(O)H.
   R¹³ is in particular C₁-C₄-alkyl.
   R¹⁴ is in particular H or C₁-C₄-alkyl.
   R¹⁵ is in particular H or C₁-C₄-alkyl.
   R¹⁶ is in particular H or C₁-C₄-alkyl.

Examples of preferred compounds of the formula I according to the invention are the enantiomers, listed in tables 1 to 60 below, of the formulae I-L and I-D, and also the racemate of the formula I-R, where the variables R¹, R², R³ and Y in each case together have the meaning given in one of rows 1 to 814 of table A: 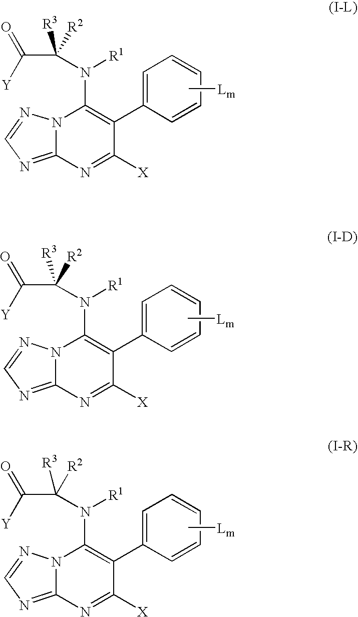
Table 1

Compounds of the formulae I-L, I-D and I-R in which X is chlorine, Lₘ is 2-fluoro-6-chloro and the combination of Y, R¹, R² and R³ for one compound corresponds in each case to one row of table A

Table 2

Compounds of the formulae I-D, I-L and I-R in which X is chlorine, Lₘ is 2,6-difluoro- and the combination of Y, R¹, R² and R³ for one compound corresponds in each case to one row of table A

Table 3

Compounds of the formulae I-D, I-L and I-R in which X is chlorine, Lₘ is 2,6-dichloro and the combination of Y, R¹, R² and R³ for one compound corresponds in each case to one row of table A

Table 4

Compounds of the formulae I-D, I-L and I-R in which X is chlorine, Lₘ is 2-fluoro-6-methyl and the combination of Y, R¹, R² and R³ for one compound corresponds in each case to one row of table A

Table 5

Compounds of the formulae I-D, I-L and I-R in which X is chlorine, Lₘ is 2,4,6-trifluoro and the combination of Y, R¹, R² and R³ for one compound corresponds in each case to one row of table A

Table 6

Compounds of the formulae I-D, I-L and I-R in which X is chlorine, Lₘ is 2,6-difluoro-4-methoxy and the combination of Y, R¹, R² and R³ for one compound corresponds in each case to one row of table A

Table 7

Compounds of the formulae I-D, I-L and I-R in which X is chlorine, Lₘ is pentafluoro and the combination of Y, R¹, R² and R³ for one compound corresponds in each case to one row of table A

Table 8

Compounds of the formulae I-D, I-L and I-R in which X is chlorine, Lₘ is 2-methyl-4-fluoro and the combination of Y, R¹, R² and R³ for one compound corresponds in each case to one row of table A

Table 9

Compounds of the formulae I-D, I-L and I-R in which X is chlorine, Lₘ is 2-trifluoromethyl and the combination of Y, R¹, R² and R³ for one compound corresponds in each case to one row of table A

Table 10

Compounds of the formulae I-D, I-L and I-R in which X is chlorine, Lₘ is 2-methoxy-6-fluoro and the combination of for one compound corresponds in each case to one row of table A

Table 11

Compounds of the formulae I-D, I-L and I-R in which X is chlorine, Lₘ is 2-chloro and the combination of Y, R¹, R² and R³ for one compound corresponds in each case to one row of table A

Table 12

Compounds of the formulae I-D, I-L and I-R in which X is chlorine, Lₘ is 2-fluoro and the combination of Y, R¹, R² and R³ for one compound corresponds in each case to one row of table A

Table 13

Compounds of the formulae I-D, I-L and I-R in which X is chlorine, Lₘ is 2,4-difluoro and the combination of Y, R¹, R² and R³ for one compound corresponds in each case to one row of table A

Table 14

Compounds of the formulae I-D, I-L and I-R in which X is chlorine, Lₘ is 2-fluoro-4-chloro and the combination of Y, R¹, R² and R³ for one compound corresponds in each case to one row of table A

Table 15

Compounds of the formulae I-D, I-L and I-R in which X is chlorine, Lₘ is 2-chloro-4-fluoro and the combination of Y, R¹, R² and R³ for one compound corresponds in each case to one row of table A

Table 16

Compounds of the formulae I-D, I-L and I-R in which X is chlorine, Lₘ is 2,3-difluoro and the combination of Y, R¹, R² and R³ for one compound corresponds in each case to one row of table A

Table 17

Compounds of the formulae I-D, I-L and I-R in which X is chlorine, Lₘ is 2,5-difluoro and the combination of Y, R¹, R² and R³ for one compound corresponds in each case to one row of table A

Table 18

Compounds of the formulae I-D, I-L and I-R in which X is chlorine, Lₘ is 2,3,4-trifluoro and the combination of Y, R¹, R² and R³ for one compound corresponds in each case to one row of table A

Table 19

Compounds of the formulae I-D, I-L and I-R in which X is chlorine, Lₘ is 2-methyl and the combination of Y, R¹, R² and R³ for one compound corresponds in each case to one row of table A

Table 20

Compounds of the formulae I-D, I-L and I-R in which X is chlorine, Lₘ is 2,4-dimethyl and the combination of Y, R¹, R² and R³ for one compound corresponds in each case to one row of table A

Table 21

Compounds of the formulae I-D, I-L and I-R in which X is chlorine, Lₘ is 2-methyl-4-chloro and the combination of Y, R¹, R² and R³ for one compound corresponds in each case to one row of table A

Table 22

Compounds of the formulae I-D, I-L and I-R in which X is chlorine, LM is 2-fluoro-4-methyl and the combination of Y, R¹, R² and R³ for one compound corresponds in each case to one row of table A

Table 23

Compounds of the formulae I-D, I-L and I-R in which X is chlorine, Lₘ is 2,6-dimethyl and the combination of Y, R¹, R² and R³ for one compound corresponds in each case to one row of table A

Table 24

Compounds of the formulae I-D, I-L and I-R in which X is chlorine, Lₘ is 2,4,6-trimethyl and the combination of Y, R¹, R² and R³ for one compound corresponds in each case to one row of table A

Table 25

Compounds of the formulae I-D, I-L and I-R in which X is chlorine, Lₘ is 2,6-difluoro-4-cyano and the combination of Y, R¹, R² and R³ for one compound corresponds in each case to one row of table A

Table 26

Compounds of the formulae I-D, I-L and I-R in which X is chlorine, Lₘ is 2,6-difluoro-4-methyl and the combination of Y, R¹, R² and R³ for one compound corresponds in each case to one row of table A

Table 27

Compounds of the formulae I-D, I-L and I-R in which X is chlorine, Lₘ is 2,6-difluoro-4-methoxycarbonyl and the combination of Y, R¹, R² and R³ for one compound corresponds in each case to one row of table A

Table 28

Compounds of the formulae I-D, I-L and I-R in which X is chlorine, Lₘ is 2-trifluoromethyl-4-fluoro and the combination of Y, R¹, R² and R³ for one compound corresponds in each case to one row of table A

Table 29

Compounds of the formulae I-D, I-L and I-R in which X is chlorine, Lₘ is 2-trifluoromethyl-5-fluoro and the combination of Y, R¹, R² and R³ for one compound corresponds in each case to one row of table A

Table 30

Compounds of the formulae I-D, I-L and I-R in which X is chlorine, Lₘ is 2-trifluoromethyl-5-chloro and the combination of Y, R¹, R² and R³ for one compound corresponds in each case to one row of table A

Table 31

Compounds of the formulae I-L, I-D and I-R in which X is methyl, Lₘ is 2-fluoro-6-chloro and the combination of Y, R¹, R² and R³ for one compound corresponds in each case to one row of table A

Table 32

Compounds of the formulae I-L, I-D and I-R in which X is methyl, Lₘ is 2,6-difluoro and the combination of Y, R¹, R² and R³ for one compound corresponds in each case to one row of table A

Table 33

Compounds of the formulae I-L, I-D and I-R in which X is methyl, Lₘ is 2,6-dichloro and the combination of Y, R¹, R² and R³ for one compound corresponds in each case to one row of table A

Table 34

Compounds of the formulae I-L, I-D and I-R in which X is methyl; Lₘ is 2-fluoro-6-methyl and the combination of Y, R¹, R² and R³ for one compound corresponds in each case to one row of table A

Table 35

Compounds of the formulae I-L, I-D and I-R in which X is methyl, Lₘ is 2,4,6-trifluoro and the combination of Y, R¹, R² and R³ for one compound corresponds in each case to one row of table A

Table 36

Compounds of the formulae I-L, I-D and I-R in which X is methyl, Lₘ is 2,6-difluoro-4-methoxy and the combination of for one compound corresponds in each case to one row of table A

Table 37

Compounds of the formulae I-L, I-D and I-R in which X is methyl, Lₘ is pentafluoro and the combination of for one compound corresponds in each case to one row of table A

Table 38

Compounds of the formulae I-L, I-D and I-R in which X is methyl, Lₘ is 2-methyl-4-fluoro and the combination of Y, R¹, R² and R³ for one compound corresponds in each case to one row of table A

Table 39

Compounds of the formulae I-L, I-D and I-R in which X is methyl, Lₘ is 2-trifluoromethyl and the combination of Y, R¹, R² and R³ for one compound corresponds in each case to one row of table A

Table 40

Compounds of the formulae I-L, I-D and I-R in which X is methyl, Lₘ is 2-methoxy-6-fluoro and the combination of Y, R¹, R² and R³ for one compound corresponds in each case to one row of table A

Table 41

Compounds of the formulae I-L, I-D and I-R in which X is methyl, Lₘ is 2-chloro and the combination of Y, R¹, R² and R³ for one compound corresponds in each case to one row of table A

Table 42

Compounds of the formulae I-L, I-D and I-R in which X is methyl, Lₘ is 2-fluoro and the combination of Y, R¹, R² and R³ for one compound corresponds in each case to one row of table A

Table 43

Compounds of the formulae I-L, I-D and I-R in which X is methyl, Lₘ is 2,4-difluoro and the combination of Y, R¹, R² and R³ for one compound corresponds in each case to one row of table A

Table 44

Compounds of the formulae I-L, I-D and I-R in which X is methyl, Lₘ is 2-fluoro-4-chloro and the combination of Y, R¹, R² and R³ for one compound corresponds in each case to one row of table A

Table 45

Compounds of the formulae I-L, I-D and I-R in which X is methyl, Lₘ is 2-chloro-4-fluoro and the combination of Y, R¹, R² and R³ for one compound corresponds in each case to one row of table A

Table 46

Compounds of the formulae I-L, I-D and I-R in which X is methyl, Lₘ is 2,3-difluoro and the combination of Y, R¹, R² and R³ for one compound corresponds in each case to one row of table A

Table 47

Compounds of the formulae I-L, I-D and I-R in which X is methyl, Lₘ is 2,5-difluoro and the combination of Y, R¹, R² and R³ for one compound corresponds in each case to one row of table A

Table 48

Compounds of the formulae I-L, I-D and I-R in which X is methyl, Lₘ is 2,3,4-trifluoro and the combination of Y, R¹, R² and R³ for one compound corresponds in each case to one row of table A

Table 49

Compounds of the formulae I-L, I-D and I-R in which X is methyl, Lₘ is 2-methyl and the combination of Y, R¹, R² and R³ for one compound corresponds in each case to one row of table A

Table 50

Compounds of the formulae I-L, I-D and I-R in which X is methyl, Lₘ is 2,4-dimethyl and the combination of Y, R¹, R² and R³ for one compound corresponds in each case to one row of table A

Table 51

Compounds of the formulae I-L, I-D and I-R in which X is methyl, Lₘ is 2-methyl-4-chloro and the combination of Y, R¹, R² and R³ for one compound corresponds in each case to one row of table A

Table 52

Compounds of the formulae I-L, I-D and I-R in which X is methyl, Lₘ is 2-fluoro-4-methyl and the combination of Y, R¹, R² and R³ for one compound corresponds in each case to one row of table A

Table 53

Compounds of the formulae I-L, I-D and I-R in which X is methyl, Lₘ is 2,6-dimethyl and the combination of Y, R¹, R² and R³ for one compound corresponds in each case to one row of table A

Table 54

Compounds of the formulae I-L, I-D and I-R in which X is methyl, Lₘ is 2,4,6-trimethyl and the combination of Y, R¹, R² and R³ for one compound corresponds in each case to one row of table A

Table 55

Compounds of the formulae I-L, I-D and I-R in which X is methyl, LM is 2,6-difluoro-4-cyano and the combination of Y, R¹, R² and R³ for one compound corresponds in each case to one row of table A

Table 56

Compounds of the formulae I-L, I-D and I-R in which X is methyl, Lₘ is 2,6-difluoro-4-methyl and the combination of Y, R¹, R² and R³ for one compound corresponds in each case to one row of table A

Table 57

Compounds of the formulae I-L, I-D and I-R in which X is methyl, Lₘ is 2,6-difluoro-4-methoxycarbonyl and the combination of Y, R¹, R² and R³ for one compound corresponds in each case to one row of table A

Table 58

Compounds of the formulae I-L, I-D and I-R in which X is methyl, Lₘ is 2-trifluoromethyl-4-fluoro and the combination of Y, R¹, R² and R³ for one compound corresponds in each case to one row of table A

Table 59

Compounds of the formulae I-L, I-D and I-R in which X is methyl, Lₘ is 2-trifluoromethyl-5-fluoro and the combination of Y, R¹, R² and R³ for one compound corresponds in each case to one row of table A

Table 60

Compounds of the formulae I-L, I-D and I-R in which X is methyl, Lₘ is 2-trifluoromethyl-5-chloro and the combination of Y, R¹, R² and R³ for one compound corresponds in each case to one row of table A

TABLE A

| No. | R¹ | R² | R³ | Y |
|---|---|---|---|---|
| 1 | H | H | H | OH |
| 2 | H | phenyl | H | OH |
| 3 | H | CH₂-indol-3-yl | H | OH |
| 4 | H | CH₂-(5-hydroxyindol-3-yl) | H | OH |
| 5 | H | CH₂-(imidazol-4-yl) | H | OH |
| 6 | H | CH₂-phenyl | H | OH |
| 7 | H | CH₂-(4-OH-phenyl) | H | OH |
| 8 | H | CH₂-cyclohexyl | H | OH |
| 9 | H | CH₃ | H | OH |
| 10 | H | CH(CH₃)₂ | H | OH |
| 11 | H | CH₂CH(CH₃)₂ | H | OH |
| 12 | H | CH(CH₃)CH₂CH₃ | H | OH |
| 13 | H | (S)—CH(CH₃)CH₂CH₃ | H | OH |
| 14 | H | (R)—CH(CH₃)CH₂CH₃ | H | OH |
| 15 | H | CH₂—SH | H | OH |
| 16 | H | CH₂—CH₂—SH | H | OH |
| 17 | H | CH₂—SCH₃ | H | OH |
| 18 | H | CH₂—CH₂—SCH₃ | H | OH |
| 19 | H | CH₂—CO—NH₂ | H | OH |
| 20 | H | CH₂—CO—OH | H | OH |
| 21 | H | CH₂—CO—OCH₃ | H | OH |
| 22 | H | CH₂—CO—OC₂H₅ | H | OH |
| 23 | H | CH₂—CO—OCH(CH₃)₂ | H | OH |
| 24 | H | CH₂—CO—OC(CH₃)₃ | H | OH |
| 25 | H | CH₂—CH₂—CO—NH₂ | H | OH |
| 26 | H | CH₂—CH₂—CO—OH | H | OH |
| 27 | H | CH₂—CH₂—CO—OCH₃ | H | OH |
| 28 | H | CH₂—CH₂—CO—OC₂H₅ | H | OH |
| 29 | H | CH₂—CH₂—CO—OCH(CH₃)₂ | H | OH |
| 30 | H | CH₂—CH₂—CO—OC(CH₃)₃ | H | OH |
| 31 | H | CH₂—OH | H | OH |
| 32 | H | CH₂—OCH₃ | H | OH |
| 33 | H | CH₂—OCH(CH₃)₂ | H | OH |
| 34 | H | CH₂—OC(CH₃)₃ | H | OH |
| 35 | H | CHCH₃—OH | H | OH |
| 36 | H | CHCH₃—OCH₃ | H | OH |
| 37 | H | CHCH₃—OCH(CH₃)₂ | H | OH |
| 38 | H | CHCH₃—OC(CH₃)₃ | H | OH |
| 39 | CH₂CH₂CH₂ | | H | OH |
| 40 | CH₂CH(OH)CH₂ | | H | OH |
| 41 | CH₂CH₂CH₂CH₂ | | H | OH |
| 42 | H | CH₂CH₂ | | OH |
| 43 | H | CH₂CH₂CH₂ | | OH |
| 44 | H | CH₂CH₂CH₂CH₂ | | OH |
| 45 | H | CH₂CH₂CH₂CH₂CH₂ | | OH |
| 46 | H | H | H | OCH₃ |
| 47 | H | phenyl | H | OCH₃ |
| 48 | H | CH₂-indol-3-yl | H | OCH₃ |
| 49 | H | CH₂-(5-hydroxyindol-3-yl) | H | OCH₃ |
| 50 | H | CH₂-(imidazol-4-yl) | H | OCH₃ |
| 51 | H | CH₂-phenyl | H | OCH₃ |
| 52 | H | CH₂-(4-OH-phenyl) | H | OCH₃ |
| 53 | H | CH₂-cyclohexyl | H | OCH₃ |
| 54 | H | CH₃ | H | OCH₃ |
| 55 | H | CH(CH₃)₂ | H | OCH₃ |
| 56 | H | CH₂CH(CH₃)₂ | H | OCH₃ |
| 57 | H | CH(CH₃)CH₂CH₃ | H | OCH₃ |
| 58 | H | (S)—CH(CH₃)CH₂CH₃ | H | OCH₃ |
| 59 | H | (R)—CH(CH₃)CH₂CH₃ | H | OCH₃ |
| 60 | H | CH₂—SH | H | OCH₃ |
| 61 | H | CH₂—CH₂—SH | H | OCH₃ |
| 62 | H | CH₂—SCH₃ | H | OCH₃ |
| 63 | H | CH₂—CH₂—SCH₃ | H | OCH₃ |
| 64 | H | CH₂—CO—NH₂ | H | OCH₃ |
| 65 | H | CH₂—CO—OH | H | OCH₃ |
| 66 | H | CH₂—CO—OCH₃ | H | OCH₃ |
| 67 | H | CH₂—CO—OC₂H₅ | H | OCH₃ |
| 68 | H | CH₂—CO—OCH(CH₃)₂ | H | OCH₃ |
| 69 | H | CH₂—CO—OC(CH₃)₃ | H | OCH₃ |
| 70 | H | CH₂—CH₂—CO—NH₂ | H | OCH₃ |
| 71 | H | CH₂—CH₂—CO—OH | H | OCH₃ |
| 72 | H | CH₂—CH₂—CO—OCH₃ | H | OCH₃ |
| 73 | H | CH₂—CH₂—CO—OC₂H₅ | H | OCH₃ |
| 74 | H | CH₂—CH₂—CO—OCH(CH₃)₂ | H | OCH₃ |
| 75 | H | CH₂—CH₂—CO—OC(CH₃)₃ | H | OCH₃ |
| 76 | H | CH₂—OH | H | OCH₃ |
| 77 | H | CH₂—OCH₃ | H | OCH₃ |
| 78 | H | CH₂—OCH(CH₃)₂ | H | OCH₃ |
| 79 | H | CH₂—OC(CH₃)₃ | H | OCH₃ |
| 80 | H | CHCH₃—OH | H | OCH₃ |
| 81 | H | CHCH₃—OCH₃ | H | OCH₃ |
| 82 | H | CHCH₃—OCH(CH₃)₂ | H | OCH₃ |
| 83 | H | CHCH₃—OC(CH₃)₃ | H | OCH₃ |
| 84 | CH₂CH₂CH₂ | | H | OCH₃ |
| 85 | CH₂CH(OH)CH₂ | | H | OCH₃ |
| 86 | CH₂CH₂CH₂CH₂ | | H | OCH₃ |
| 87 | H | CH₂CH₂ | | OCH₃ |
| 88 | H | CH₂CH₂CH₂ | | OCH₃ |
| 89 | H | CH₂CH₂CH₂CH₂ | | OCH₃ |
| 90 | H | CH₂CH₂CH₂CH₂CH₂ | | OCH₃ |
| 91 | H | H | H | OCH₂CH₃ |
| 92 | H | phenyl | H | OCH₂CH₃ |
| 93 | H | CH₂-indol-3-yl | H | OCH₂CH₃ |
| 94 | H | CH₂-(5-hydroxyindol-3-yl) | H | OCH₂CH₃ |
| 95 | H | CH₂-(imidazol-4-yl) | H | OCH₂CH₃ |
| 96 | H | CH₂-phenyl | H | OCH₂CH₃ |
| 97 | H | CH₂-(4-OH-phenyl) | H | OCH₂CH₃ |
| 98 | H | CH₂-cyclohexyl | H | OCH₂CH₃ |
| 99 | H | CH₃ | H | OCH₂CH₃ |
| 100 | H | CH(CH₃)₂ | H | OCH₂CH₃ |
| 101 | H | CH₂CH(CH₃)₂ | H | OCH₂CH₃ |
| 102 | H | CH(CH₃)CH₂CH₃ | H | OCH₂CH₃ |
| 103 | H | (S)—CH(CH₃)CH₂CH₃ | H | OCH₂CH₃ |
| 104 | H | (R)—CH(CH₃)CH₂CH₃ | H | OCH₂CH₃ |
| 105 | H | CH₂—SH | H | OCH₂CH₃ |
| 106 | H | CH₂—CH₂—SH | H | OCH₂CH₃ |
| 107 | H | CH₂—SCH₃ | H | OCH₂CH₃ |
| 108 | H | CH₂—CH₂—SCH₃ | H | OCH₂CH₃ |
| 109 | H | CH₂—CO—NH₂ | H | OCH₂CH₃ |
| 110 | H | CH₂—CO—OH | H | OCH₂CH₃ |
| 111 | H | CH₂—CO—OCH₃ | H | OCH₂CH₃ |
| 112 | H | CH₂—CO—OC₂H₅ | H | OCH₂CH₃ |
| 113 | H | CH₂—CO—OCH(CH₃)₂ | H | OCH₂CH₃ |
| 114 | H | CH₂—CO—OC(CH₃)₃ | H | OCH₂CH₃ |
| 115 | H | CH₂—CH₂—CO—NH₂ | H | OCH₂CH₃ |
| 116 | H | CH₂—CH₂—CO—OH | H | OCH₂CH₃ |
| 117 | H | CH₂—CH₂—CO—OCH₃ | H | OCH₂CH₃ |
| 118 | H | CH₂—CH₂—CO—OC₂H₅ | H | OCH₂CH₃ |
| 119 | H | CH₂—CH₂—CO—OCH(CH₃)₂ | H | OCH₂CH₃ |
| 120 | H | CH₂—CH₂—CO—OC(CH₃)₃ | H | OCH₂CH₃ |
| 121 | H | CH₂—OH | H | OCH₂CH₃ |
| 122 | H | CH₂—OCH₃ | H | OCH₂CH₃ |
| 123 | H | CH₂—OCH(CH₃)₂ | H | OCH₂CH₃ |
| 124 | H | CH₂—OC(CH₃)₃ | H | OCH₂CH₃ |
| 125 | H | CHCH₃—OH | H | OCH₂CH₃ |
| 126 | H | CHCH₃—OCH₃ | H | OCH₂CH₃ |
| 127 | H | CHCH₃—OCH(CH₃)₂ | H | OCH₂CH₃ |
| 128 | H | CHCH₃—OC(CH₃)₃ | H | OCH₂CH₃ |
| 129 | CH₂CH₂CH₂ | | H | OCH₂CH₃ |
| 130 | CH₂CH(OH)CH₂ | | H | OCH₂CH₃ |
| 131 | CH₂CH₂CH₂CH₂ | | H | OCH₂CH₃ |
| 132 | H | CH₂CH₂ | | OCH₂CH₃ |
| 133 | H | CH₂CH₂CH₂ | | OCH₂CH₃ |
| 134 | H | CH₂CH₂CH₂CH₂ | | OCH₂CH₃ |
| 135 | H | CH₂CH₂CH₂CH₂CH₂ | | OCH₂CH₃ |
| 136 | H | H | H | OCH₂CH₂CH₃ |
| 137 | H | phenyl | H | OCH₂CH₂CH₃ |
| 138 | H | CH₂-indol-3-yl | H | OCH₂CH₂CH₃ |
| 139 | H | CH₂-(5-hydroxyindol-3-yl) | H | OCH₂CH₂CH₃ |
| 140 | H | CH₂-(imidazol-4-yl) | H | OCH₂CH₂CH₃ |
| 141 | H | CH₂-phenyl | H | OCH₂CH₂CH₃ |
| 142 | H | CH₂-(4-OH-phenyl) | H | OCH₂CH₂CH₃ |
| 143 | H | CH₂-cyclohexyl | H | OCH₂CH₂CH₃ |
| 144 | H | CH₃ | H | OCH₂CH₂CH₃ |
| 145 | H | CH(CH₃)₂ | H | OCH₂CH₂CH₃ |
| 146 | H | CH₂CH(CH₃)₂ | H | OCH₂CH₂CH₃ |
| 147 | H | CH(CH₃)CH₂CH₃ | H | OCH₂CH₂CH₃ |
| 148 | H | (S)—CH(CH₃)CH₂CH₃ | H | OCH₂CH₂CH₃ |
| 149 | H | (R)—CH(CH₃)CH₂CH₃ | H | OCH₂CH₂CH₃ |
| 150 | H | CH₂—SH | H | OCH₂CH₂CH₃ |
| 151 | H | CH₂—CH₂—SH | H | OCH₂CH₂CH₃ |
| 152 | H | CH₂—SCH₃ | H | OCH₂CH₂CH₃ |
| 153 | H | CH₂—CH₂—SCH₃ | H | OCH₂CH₂CH₃ |
| 154 | H | CH₂—CO—NH₂ | H | OCH₂CH₂CH₃ |
| 155 | H | CH₂—CO—OH | H | OCH₂CH₂CH₃ |
| 156 | H | CH₂—CO—OCH₃ | H | OCH₂CH₂CH₃ |
| 157 | H | CH₂—CO—OC₂H₅ | H | OCH₂CH₂CH₃ |
| 158 | H | CH₂—CO—OCH(CH₃)₂ | H | OCH₂CH₂CH₃ |
| 159 | H | CH₂—CO—OC(CH₃)₃ | H | OCH₂CH₂CH₃ |
| 160 | H | CH₂—CH₂—CO—NH₂ | H | OCH₂CH₂CH₃ |
| 161 | H | CH₂—CH₂—CO—OH | H | OCH₂CH₂CH₃ |
| 162 | H | CH₂—CH₂—CO—OCH₃ | H | OCH₂CH₂CH₃ |
| 163 | H | CH₂—CH₂—CO—OC₂H₅ | H | OCH₂CH₂CH₃ |
| 164 | H | CH₂—CH₂—CO—OCH(CH₃)₂ | H | OCH₂CH₂CH₃ |
| 165 | H | CH₂—CH₂—CO—OC(CH₃)₃ | H | OCH₂CH₂CH₃ |
| 166 | H | CH₂—OH | H | OCH₂CH₂CH₃ |
| 167 | H | CH₂—OCH₃ | H | OCH₂CH₂CH₃ |
| 168 | H | CH₂—OCH(CH₃)₂ | H | OCH₂CH₂CH₃ |
| 169 | H | CH₂—OC(CH₃)₃ | H | OCH₂CH₂CH₃ |
| 170 | H | CHCH₃—OH | H | OCH₂CH₂CH₃ |
| 171 | H | CHCH₃—OCH₃ | H | OCH₂CH₂CH₃ |
| 172 | H | CHCH₃—OCH(CH₃)₂ | H | OCH₂CH₂CH₃ |
| 173 | H | CHCH₃—OC(CH₃)₃ | H | OCH₂CH₂CH₃ |
| 174 | CH₂CH₂CH₂ | | H | OCH₂CH₂CH₃ |
| 175 | CH₂CH(OH)CH₂ | | H | OCH₂CH₂CH₃ |
| 176 | CH₂CH₂CH₂CH₂ | | H | OCH₂CH₂CH₃ |
| 177 | H | CH₂CH₂ | | OCH₂CH₂CH₃ |
| 178 | H | CH₂CH₂CH₂ | | OCH₂CH₂CH₃ |
| 179 | H | CH₂CH₂CH₂CH₂ | | OCH₂CH₂CH₃ |
| 180 | H | CH₂CH₂CH₂CH₂CH₂ | | OCH₂CH₂CH₃ |
| 181 | H | H | H | OCH₂CH₂OCH₃ |
| 182 | H | phenyl | H | OCH₂CH₂OCH₃ |
| 183 | H | CH₂-indol-3-yl | H | OCH₂CH₂OCH₃ |
| 184 | H | CH₂-(5-hydroxyindol-3-yl) | H | OCH₂CH₂OCH₃ |
| 185 | H | CH₂-(imidazol-4-yl) | H | OCH₂CH₂OCH₃ |
| 186 | H | CH₂-phenyl | H | OCH₂CH₂OCH₃ |
| 187 | H | CH₂-(4-OH-phenyl) | H | OCH₂CH₂OCH₃ |
| 188 | H | CH₂-cyclohexyl | H | OCH₂CH₂OCH₃ |
| 189 | H | CH₃ | H | OCH₂CH₂OCH₃ |
| 190 | H | CH(CH₃)₂ | H | OCH₂CH₂OCH₃ |
| 191 | H | CH₂CH(CH₃)₂ | H | OCH₂CH₂OCH₃ |
| 192 | H | CH(CH₃)CH₂CH₃ | H | OCH₂CH₂OCH₃ |
| 193 | H | (S)—CH(CH₃)CH₂CH₃ | H | OCH₂CH₂OCH₃ |
| 194 | H | (R)—CH(CH₃)CH₂CH₃ | H | OCH₂CH₂OCH₃ |
| 195 | H | CH₂—SH | H | OCH₂CH₂OCH₃ |
| 196 | H | CH₂—CH₂—SH | H | OCH₂CH₂OCH₃ |
| 197 | H | CH₂—SCH₃ | H | OCH₂CH₂OCH₃ |
| 198 | H | CH₂—CH₂—SCH₃ | H | OCH₂CH₂OCH₃ |
| 199 | H | CH₂—CO—NH₂ | H | OCH₂CH₂OCH₃ |
| 200 | H | CH₂—CO—OH | H | OCH₂CH₂OCH₃ |
| 201 | H | CH₂—CO—OCH₃ | H | OCH₂CH₂OCH₃ |
| 202 | H | CH₂—CO—OC₂H₅ | H | OCH₂CH₂OCH₃ |
| 203 | H | CH₂—CO—OCH(CH₃)₂ | H | OCH₂CH₂OCH₃ |
| 204 | H | CH₂—CO—OC(CH₃)₃ | H | OCH₂CH₂OCH₃ |
| 205 | H | CH₂—CH₂—CO—NH₂ | H | OCH₂CH₂OCH₃ |
| 206 | H | CH₂—CH₂—CO—OH | H | OCH₂CH₂OCH₃ |
| 207 | H | CH₂—CH₂—CO—OCH₃ | H | OCH₂CH₂OCH₃ |
| 208 | H | CH₂—CH₂—CO—OC₂H₅ | H | OCH₂CH₂OCH₃ |
| 209 | H | CH₂—CH₂—CO—OCH(CH₃)₂ | H | OCH₂CH₂OCH₃ |
| 210 | H | CH₂—CH₂—CO—OC(CH₃)₃ | H | OCH₂CH₂OCH₃ |
| 211 | H | CH₂—OH | H | OCH₂CH₂OCH₃ |
| 212 | H | CH₂—OCH₃ | H | OCH₂CH₂OCH₃ |
| 213 | H | CH₂—OCH(CH₃)₂ | H | OCH₂CH₂OCH₃ |
| 214 | H | CH₂—OC(CH₃)₃ | H | OCH₂CH₂OCH₃ |
| 215 | H | CHCH₃—OH | H | OCH₂CH₂OCH₃ |
| 216 | H | CHCH₃—OCH₃ | H | OCH₂CH₂OCH₃ |
| 217 | H | CHCH₃—OCH(CH₃)₂ | H | OCH₂CH₂OCH₃ |
| 218 | H | CHCH₃—OC(CH₃)₃ | H | OCH₂CH₂OCH₃ |
| 219 | CH₂CH₂CH₂ | | H | OCH₂CH₂OCH₃ |
| 220 | CH₂CH(OH)CH₂ | | H | OCH₂CH₂OCH₃ |
| 221 | CH₂CH₂CH₂CH₂ | | H | OCH₂CH₂OCH₃ |
| 222 | H | CH₂CH₂ | | OCH₂CH₂OCH₃ |
| 223 | H | CH₂CH₂CH₂ | | OCH₂CH₂OCH₃ |
| 224 | H | CH₂CH₂CH₂CH₂ | | OCH₂CH₂OCH₃ |
| 225 | H | CH₂CH₂CH₂CH₂CH₂ | | OCH₂CH₂OCH₃ |
| 226 | H | H | H | OC(CH₃)₃ |
| 227 | H | phenyl | H | OC(CH₃)₃ |
| 228 | H | CH₂-indol-3-yl | H | OC(CH₃)₃ |
| 229 | H | CH₂-(5-hydroxyindol-3-yl) | H | OC(CH₃)₃ |
| 230 | H | CH₂-(imidazol-4-yl) | H | OC(CH₃)₃ |
| 231 | H | CH₂-phenyl | H | OC(CH₃)₃ |
| 232 | H | CH₂-(4-OH-phenyl) | H | OC(CH₃)₃ |
| 233 | H | CH₂-cyclohexyl | H | OC(CH₃)₃ |
| 234 | H | CH₃ | H | OC(CH₃)₃ |
| 235 | H | CH(CH₃)₂ | H | OC(CH₃)₃ |
| 236 | H | CH₂CH(CH₃)₂ | H | OC(CH₃)₃ |
| 237 | H | CH(CH₃)CH₂CH₃ | H | OC(CH₃)₃ |
| 238 | H | (S)—CH(CH₃)CH₂CH₃ | H | OC(CH₃)₃ |
| 239 | H | (R)—CH(CH₃)CH₂CH₃ | H | OC(CH₃)₃ |
| 240 | H | CH₂—SH | H | OC(CH₃)₃ |
| 241 | H | CH₂—CH₂—SH | H | OC(CH₃)₃ |
| 242 | H | CH₂—SCH₃ | H | OC(CH₃)₃ |
| 243 | H | CH₂—CH₂—SCH₃ | H | OC(CH₃)₃ |
| 244 | H | CH₂—CO—NH₂ | H | OC(CH₃)₃ |
| 245 | H | CH₂—CO—OH | H | OC(CH₃)₃ |
| 246 | H | CH₂—CO—OCH₃ | H | OC(CH₃)₃ |
| 247 | H | CH₂—CO—OC₂H₅ | H | OC(CH₃)₃ |
| 248 | H | CH₂—CO—OCH(CH₃)₂ | H | OC(CH₃)₃ |
| 249 | H | CH₂—CO—OC(CH₃)₃ | H | OC(CH₃)₃ |
| 250 | H | CH₂—CH₂—CO—NH₂ | H | OC(CH₃)₃ |
| 251 | H | CH₂—CH₂—CO—OH | H | OC(CH₃)₃ |
| 252 | H | CH₂—CH₂—CO—OCH₃ | H | OC(CH₃)₃ |
| 253 | H | CH₂—CH₂—CO—OC₂H₅ | H | OC(CH₃)₃ |
| 254 | H | CH₂—CH₂—CO—OCH(CH₃)₂ | H | OC(CH₃)₃ |
| 255 | H | CH₂—CH₂—CO—OC(CH₃)₃ | H | OC(CH₃)₃ |
| 256 | H | CH₂—OH | H | OC(CH₃)₃ |
| 257 | H | CH₂—OCH₃ | H | OC(CH₃)₃ |
| 258 | H | CH₂—OCH(CH₃)₂ | H | OC(CH₃)₃ |
| 259 | H | CH₂—OC(CH₃)₃ | H | OC(CH₃)₃ |
| 260 | H | CHCH₃—OH | H | OC(CH₃)₃ |
| 261 | H | CHCH₃—OCH₃ | H | OC(CH₃)₃ |
| 262 | H | CHCH₃—OCH(CH₃)₂ | H | OC(CH₃)₃ |
| 263 | H | CHCH₃—OC(CH₃)₃ | H | OC(CH₃)₃ |
| 264 | CH₂CH₂CH₂ | | H | OC(CH₃)₃ |
| 265 | CH₂CH(OH)CH₂ | | H | OC(CH₃)₃ |
| 266 | CH₂CH₂CH₂CH₂ | | H | OC(CH₃)₃ |
| 267 | H | CH₂CH₂ | | OC(CH₃)₃ |
| 268 | H | CH₂CH₂CH₂ | | OC(CH₃)₃ |
| 269 | H | CH₂CH₂CH₂CH₂ | | OC(CH₃)₃ |
| 270 | H | CH₂CH₂CH₂CH₂CH₂ | | OC(CH₃)₃ |
| 271 | H | H | H | OCH(CH₃)₂ |
| 272 | H | phenyl | H | OCH(CH₃)₂ |
| 273 | H | CH₂-indol-3-yl | H | OCH(CH₃)₂ |
| 274 | H | CH₂-(5-hydroxyindol-3-yl) | H | OCH(CH₃)₂ |
| 275 | H | CH₂-(imidazol-4-yl) | H | OCH(CH₃)₂ |
| 276 | H | CH₂-phenyl | H | OCH(CH₃)₂ |
| 277 | H | CH₂-(4-OH-phenyl) | H | OCH(CH₃)₂ |
| 278 | H | CH₂-cyclohexyl | H | OCH(CH₃)₂ |
| 279 | H | CH₃ | H | OCH(CH₃)₂ |
| 280 | H | CH(CH₃)₂ | H | OCH(CH₃)₂ |
| 281 | H | CH₂CH(CH₃)₂ | H | OCH(CH₃)₂ |
| 282 | H | CH(CH₃)CH₂CH₃ | H | OCH(CH₃)₂ |
| 283 | H | (S)—CH(CH₃)CH₂CH₃ | H | OCH(CH₃)₂ |
| 284 | H | (R)—CH(CH₃)CH₂CH₃ | H | OCH(CH₃)₂ |
| 285 | H | CH₂—SH | H | OCH(CH₃)₂ |
| 286 | H | CH₂—CH₂—SH | H | OCH(CH₃)₂ |
| 287 | H | CH₂—SCH₃ | H | OCH(CH₃)₂ |
| 288 | H | CH₂—CH₂—SCH₃ | H | OCH(CH₃)₂ |
| 289 | H | CH₂—CO—NH₂ | H | OCH(CH₃)₂ |
| 290 | H | CH₂—CO—OH | H | OCH(CH₃)₂ |
| 291 | H | CH₂—CO—OCH₃ | H | OCH(CH₃)₂ |
| 292 | H | CH₂—CO—OC₂H₅ | H | OCH(CH₃)₂ |
| 293 | H | CH₂—CO—OCH(CH₃)₂ | H | OCH(CH₃)₂ |
| 294 | H | CH₂—CO—OC(CH₃)₃ | H | OCH(CH₃)₂ |
| 295 | H | CH₂—CH₂—CO—NH₂ | H | OCH(CH₃)₂ |
| 296 | H | CH₂—CH₂—CO—OH | H | OCH(CH₃)₂ |
| 297 | H | CH₂—CH₂—CO—OCH₃ | H | OCH(CH₃)₂ |
| 298 | H | CH₂—CH₂—CO—OC₂H₅ | H | OCH(CH₃)₂ |
| 299 | H | CH₂—CH₂—CO—OCH(CH₃)₂ | H | OCH(CH₃)₂ |
| 300 | H | CH₂—CH₂—CO—OC(CH₃)₃ | H | OCH(CH₃)₂ |
| 301 | H | CH₂—OH | H | OCH(CH₃)₂ |
| 302 | H | CH₂—OCH₃ | H | OCH(CH₃)₂ |
| 303 | H | CH₂—OCH(CH₃)₂ | H | OCH(CH₃)₂ |
| 304 | H | CH₂—OC(CH₃)₃ | H | OCH(CH₃)₂ |
| 305 | H | CHCH₃—OH | H | OCH(CH₃)₂ |
| 306 | H | CHCH₃—OCH₃ | H | OCH(CH₃)₂ |
| 307 | H | CHCH₃—OCH(CH₃)₂ | H | OCH(CH₃)₂ |
| 308 | H | CHCH₃—OC(CH₃)₃ | H | OCH(CH₃)₂ |
| 309 | CH₂CH₂CH₂ | | H | OCH(CH₃)₂ |
| 310 | CH₂CH(OH)CH₂ | | H | OCH(CH₃)₂ |
| 311 | CH₂CH₂CH₂CH₂ | | H | OCH(CH₃)₂ |
| 312 | H | CH₂CH₂ | | OCH(CH₃)₂ |
| 313 | H | CH₂CH₂CH₂ | | OCH(CH₃)₂ |
| 314 | H | CH₂CH₂CH₂CH₂ | | OCH(CH₃)₂ |
| 315 | H | CH₂CH₂CH₂CH₂CH₂ | | OCH(CH₃)₂ |
| 316 | H | H | H | OCH₂CH═CH₂ |
| 317 | H | phenyl | H | OCH₂CH═CH₂ |
| 318 | H | CH₂-indol-3-yl | H | OCH₂CH═CH₂ |
| 319 | H | CH₂-(5-hydroxyindol-3-yl) | H | OCH₂CH═CH₂ |
| 320 | H | CH₂-(imidazol-4-yl) | H | OCH₂CH═CH₂ |
| 321 | H | CH₂-phenyl | H | OCH₂CH═CH₂ |
| 322 | H | CH₂-(4-OH-phenyl) | H | OCH₂CH═CH₂ |
| 323 | H | CH₂-cyclohexyl | H | OCH₂CH═CH₂ |
| 324 | H | CH₃ | H | OCH₂CH═CH₂ |
| 325 | H | CH(CH₃)₂ | H | OCH₂CH═CH₂ |
| 326 | H | CH₂CH(CH₃)₂ | H | OCH₂CH═CH₂ |
| 327 | H | CH(CH₃)CH₂CH₃ | H | OCH₂CH═CH₂ |
| 328 | H | (S)—CH(CH₃)CH₂CH₃ | H | OCH₂CH═CH₂ |
| 329 | H | (R)—CH(CH₃)CH₂CH₃ | H | OCH₂CH═CH₂ |
| 330 | H | CH₂—SH | H | OCH₂CH═CH₂ |
| 331 | H | CH₂—CH₂—SH | H | OCH₂CH═CH₂ |
| 332 | H | CH₂—SCH₃ | | OCH₂CH═CH₂ |
| 333 | H | CH₂—CH₂—SCH₃ | H | OCH₂CH═CH₂ |
| 334 | H | CH₂—CO—NH₂ | H | OCH₂CH═CH₂ |
| 335 | H | CH₂—CO—OH | H | OCH₂CH═CH₂ |
| 336 | H | CH₂—CO—OCH₃ | H | OCH₂CH═CH₂ |
| 337 | H | CH₂—CO—OC₂H₅ | H | OCH₂CH═CH₂ |
| 338 | H | CH₂—CO—OCH(CH₃)₂ | H | OCH₂CH═CH₂ |
| 339 | H | CH₂—CO—OC(CH₃)₃ | H | OCH₂CH═CH₂ |
| 340 | H | CH₂—CH₂—CO—NH₂ | H | OCH₂CH═CH₂ |
| 341 | H | CH₂—CH₂—CO—OH | H | OCH₂CH═CH₂ |
| 342 | H | CH₂—CH₂—CO—OCH₃ | H | OCH₂CH═CH₂ |
| 343 | H | CH₂—CH₂—CO—OC₂H₅ | H | OCH₂CH═CH₂ |
| 344 | H | CH₂—CH₂—CO—OCH(CH₃)₂ | H | OCH₂CH═CH₂ |
| 345 | H | CH₂—CH₂—CO—OC(CH₃)₃ | H | OCH₂CH═CH₂ |
| 346 | H | CH₂—OH | H | OCH₂CH═CH₂ |
| 347 | H | CH₂—OCH₃ | H | OCH₂CH═CH₂ |
| 348 | H | CH₂—OCH(CH₃)₂ | H | OCH₂CH═CH₂ |
| 349 | H | CH₂—OC(CH₃)₃ | H | OCH₂CH═CH₂ |
| 350 | H | CHCH₃—OH | H | OCH₂CH═CH₂ |
| 351 | H | CHCH₃—OCH₃ | H | OCH₂CH═CH₂ |
| 352 | H | CHCH₃—OCH(CH₃)₂ | H | OCH₂CH═CH₂ |
| 353 | H | CHCH₃—OC(CH₃)₃ | H | OCH₂CH═CH₂ |
| 354 | CH₂CH₂CH₂ | | H | OCH₂CH═CH₂ |
| 355 | CH₂CH(OH)CH₂ | | H | OCH₂CH═CH₂ |
| 356 | CH₂CH₂CH₂CH₂ | | H | OCH₂CH═CH₂ |
| 357 | H | CH₂CH₂ | | OCH₂CH═CH₂ |
| 358 | H | CH₂CH₂CH₂ | | OCH₂CH═CH₂ |
| 359 | H | CH₂CH₂CH₂CH₂ | | OCH₂CH═CH₂ |
| 360 | H | CH₂CH₂CH₂CH₂CH₂ | | OCH₂CH═CH₂ |
| 361 | H | H | H | NHCH₃ |
| 362 | H | phenyl | H | NHCH₃ |
| 363 | H | CH₂-indol-3-yl | H | NHCH₃ |
| 364 | H | CH₂-(5-hydroxyindol-3-yl) | H | NHCH₃ |
| 365 | H | CH₂-(imidazol-4-yl) | H | NHCH₃ |
| 366 | H | CH₂-phenyl | H | NHCH₃ |
| 367 | H | CH₂-(4-OH-phenyl) | H | NHCH₃ |
| 368 | H | CH₂-cyclohexyl | H | NHCH₃ |
| 369 | H | CH₃ | H | NHCH₃ |
| 370 | H | CH(CH₃)₂ | H | NHCH₃ |
| 371 | H | CH₂CH(CH₃)₂ | H | NHCH₃ |
| 372 | H | CH(CH₃)CH₂CH₃ | H | NHCH₃ |
| 373 | H | (S)—CH(CH₃)CH₂CH₃ | H | NHCH₃ |
| 374 | H | (R)—CH(CH₃)CH₂CH₃ | H | NHCH₃ |
| 375 | H | CH₂—SH | H | NHCH₃ |
| 376 | H | CH₂—CH₂—SH | H | NHCH₃ |
| 377 | H | CH₂—SCH₃ | H | NHCH₃ |
| 378 | H | CH₂—CH₂—SCH₃ | H | NHCH₃ |
| 379 | H | CH₂—CO—NH₂ | H | NHCH₃ |
| 380 | H | CH₂—CO—OH | H | NHCH₃ |
| 381 | H | CH₂—CO—OCH₃ | H | NHCH₃ |
| 382 | H | CH₂—CO—OC₂H₅ | H | NHCH₃ |
| 383 | H | CH₂—CO—OCH(CH₃)₂ | H | NHCH₃ |
| 384 | H | CH₂—CO—OC(CH₃)₃ | H | NHCH₃ |
| 385 | H | CH₂—CH₂—CO—NH₂ | H | NHCH₃ |
| 386 | H | CH₂—CH₂—CO—OH | H | NHCH₃ |
| 387 | H | CH₂—CH₂—CO—OCH₃ | H | NHCH₃ |
| 388 | H | CH₂—CH₂—CO—OC₂H₅ | H | NHCH₃ |
| 389 | H | CH₂—CH₂—CO—OCH(CH₃)₂ | H | NHCH₃ |
| 390 | H | CH₂—CH₂—CO—OC(CH₃)₃ | H | NHCH₃ |
| 391 | H | CH₂—OH | H | NHCH₃ |
| 392 | H | CH₂—OCH₃ | H | NHCH₃ |
| 393 | H | CH₂—OCH(CH₃)₂ | H | NHCH₃ |
| 394 | H | CH₂—OC(CH₃)₃ | H | NHCH₃ |
| 395 | H | CHCH₃—OH | H | NHCH₃ |
| 396 | H | CHCH₃—OCH₃ | H | NHCH₃ |
| 397 | H | CHCH₃—OCH(CH₃)₂ | H | NHCH₃ |
| 398 | H | CHCH₃—OC(CH₃)₃ | H | NHCH₃ |
| 399 | CH₂CH₂CH₂ | | H | NHCH₃ |
| 400 | CH₂CH(OH)CH₂ | | H | NHCH₃ |
| 401 | CH₂CH₂CH₂CH₂ | | H | NHCH₃ |
| 402 | H | CH₂CH₂ | | NHCH₃ |
| 403 | H | CH₂CH₂CH₂ | | NHCH₃ |
| 404 | H | CH₂CH₂CH₂CH₂ | | NHCH₃ |
| 405 | H | CH₂CH₂CH₂CH₂CH₂ | | NHCH₃ |
| 406 | CH₃ | H | H | OCH₃ |
| 407 | CH₃ | phenyl | H | OCH₃ |
| 408 | CH₃ | CH₂-indol-3-yl | H | OCH₃ |
| 409 | CH₃ | CH₂-(5-hydroxyindol-3-yl) | H | OCH₃ |
| 410 | CH₃ | CH₂-(imidazol-4-yl) | H | OCH₃ |
| 411 | CH₃ | CH₂-phenyl | H | OCH₃ |
| 412 | CH₃ | CH₂-(4-OH-phenyl) | H | OCH₃ |
| 413 | CH₃ | CH₂-cyclohexyl | H | OCH₃ |
| 414 | CH₃ | CH₃ | H | OCH₃ |
| 415 | CH₃ | CH(CH₃)₂ | H | OCH₃ |
| 416 | CH₃ | CH₂CH(CH₃)₂ | H | OCH₃ |
| 417 | CH₃ | CH(CH₃)CH₂CH₃ | H | OCH₃ |
| 418 | CH₃ | (S)—CH(CH₃)CH₂CH₃ | H | OCH₃ |
| 419 | CH₃ | (R)—CH(CH₃)CH₂CH₃ | H | OCH₃ |
| 420 | CH₃ | CH₂—SH | H | OCH₃ |
| 421 | CH₃ | CH₂—CH₂—SH | H | OCH₃ |
| 422 | CH₃ | CH₂—SCH₃ | H | OCH₃ |
| 423 | CH₃ | CH₂—CH₂—SCH₃ | H | OCH₃ |
| 424 | CH₃ | CH₂—CO—NH₂ | H | OCH₃ |
| 425 | CH₃ | CH₂—CO—OH | H | OCH₃ |
| 426 | CH₃ | CH₂—CO—OCH₃ | H | OCH₃ |
| 427 | CH₃ | CH₂—CO—OC₂H₅ | H | OCH₃ |
| 428 | CH₃ | CH₂—CO—OCH(CH₃)₂ | H | OCH₃ |
| 429 | CH₃ | CH₂—CO—OC(CH₃)₃ | H | OCH₃ |
| 430 | CH₃ | CH₂—CH₂—CO—NH₂ | H | OCH₃ |
| 431 | CH₃ | CH₂—CH₂—CO—OH | H | OCH₃ |
| 432 | CH₃ | CH₂—CH₂—CO—OCH₃ | H | OCH₃ |
| 433 | CH₃ | CH₂—CH₂—CO—OC₂H₅ | H | OCH₃ |
| 434 | CH₃ | CH₂—CH₂—CO—OCH(CH₃)₂ | H | OCH₃ |
| 435 | CH₃ | CH₂—CH₂—CO—OC(CH₃)₃ | H | OCH₃ |
| 436 | CH₃ | CH₂—OH | H | OCH₃ |
| 437 | CH₃ | CH₂—OCH₃ | H | OCH₃ |
| 438 | CH₃ | CH₂—OCH(CH₃)₂ | H | OCH₃ |
| 439 | CH₃ | CH₂—OC(CH₃)₃ | H | OCH₃ |
| 440 | CH₃ | CHCH₃—OH | H | OCH₃ |
| 441 | CH₃ | CHCH₃—OCH₃ | H | OCH₃ |
| 442 | CH₃ | CHCH₃—OCH(CH₃)₂ | H | OCH₃ |
| 443 | CH₃ | CHCH₃—OC(CH₃)₃ | H | OCH₃ |
| 444 | CH₃ | CH₂CH₂ | | OCH₃ |
| 445 | CH₃ | CH₂CH₂CH₂ | | OCH₃ |
| 446 | CH₃ | CH₂CH₂CH₂CH₂ | | OCH₃ |
| 447 | CH₃ | CH₂CH₂CH₂CH₂CH₂ | | OCH₃ |
| 448 | CH₃ | H | H | OCH₂CH₃ |
| 449 | CH₃ | phenyl | H | OCH₂CH₃ |
| 450 | CH₃ | CH₂-indol-3-yl | H | OCH₂CH₃ |
| 451 | CH₃ | CH₂-(5-hydroxyindol-3-yl) | H | OCH₂CH₃ |
| 452 | CH₃ | CH₂-(imidazol-4-yl) | H | OCH₂CH₃ |
| 453 | CH₃ | CH₂-phenyl | H | OCH₂CH₃ |
| 454 | CH₃ | CH₂-(4-OH-phenyl) | H | OCH₂CH₃ |
| 455 | CH₃ | CH₂-cyclohexyl | H | OCH₂CH₃ |
| 456 | CH₃ | CH₃ | H | OCH₂CH₃ |
| 457 | CH₃ | CH(CH₃)₂ | H | OCH₂CH₃ |
| 458 | CH₃ | CH₂CH(CH₃)₂ | H | OCH₂CH₃ |
| 459 | CH₃ | CH(CH₃)CH₂CH₃ | H | OCH₂CH₃ |
| 460 | CH₃ | (S)—CH(CH₃)CH₂CH₃ | H | OCH₂CH₃ |
| 461 | CH₃ | (R)—CH(CH₃)CH₂CH₃ | H | OCH₂CH₃ |
| 462 | CH₃ | CH₂—SH | H | OCH₂CH₃ |
| 463 | CH₃ | CH₂—CH₂—SH | H | OCH₂CH₃ |
| 464 | CH₃ | CH₂—SCH₃ | H | OCH₂CH₃ |
| 465 | CH₃ | CH₂—CH₂—SCH₃ | H | OCH₂CH₃ |
| 466 | CH₃ | CH₂—CO—NH₂ | H | OCH₂CH₃ |
| 467 | CH₃ | CH₂—CO—OH | H | OCH₂CH₃ |
| 468 | CH₃ | CH₂—CO—OCH₃ | H | OCH₂CH₃ |
| 469 | CH₃ | CH₂—CO—OC₂H₅ | H | OCH₂CH₃ |
| 470 | CH₃ | CH₂—CO—OCH(CH₃)₂ | H | OCH₂CH₃ |
| 471 | CH₃ | CH₂—CO—OC(CH₃)₃ | H | OCH₂CH₃ |
| 472 | CH₃ | CH₂—CH₂—CO—NH₂ | H | OCH₂CH₃ |
| 473 | CH₃ | CH₂—CH₂—CO—OH | H | OCH₂CH₃ |
| 474 | CH₃ | CH₂—CH₂—CO—OCH₃ | H | OCH₂CH₃ |
| 475 | CH₃ | CH₂—CH₂—CO—OC₂H₅ | H | OCH₂CH₃ |
| 476 | CH₃ | CH₂—CH₂—CO—OCH(CH₃)₂ | H | OCH₂CH₃ |
| 477 | CH₃ | CH₂—CH₂—CO—OC(CH₃)₃ | H | OCH₂CH₃ |
| 478 | CH₃ | CH₂—OH | H | OCH₂CH₃ |
| 479 | CH₃ | CH₂—OCH₃ | H | OCH₂CH₃ |
| 480 | CH₃ | CH₂—OCH(CH₃)₂ | H | OCH₂CH₃ |
| 481 | CH₃ | CH₂—OC(CH₃)₃ | H | OCH₂CH₃ |
| 482 | CH₃ | CHCH₃—OH | H | OCH₂CH₃ |
| 483 | CH₃ | CHCH₃—OCH₃ | H | OCH₂CH₃ |
| 484 | CH₃ | CHCH₃—OCH(CH₃)₂ | H | OCH₂CH₃ |
| 485 | CH₃ | CHCH₃—OC(CH₃)₃ | H | OCH₂CH₃ |
| 486 | CH₃ | CH₂CH₂ | | OCH₂CH₃ |
| 487 | CH₃ | CH₂CH₂CH₂ | | OCH₂CH₃ |
| 488 | CH₃ | CH₂CH₂CH₂CH₂ | | OCH₂CH₃ |
| 489 | CH₃ | CH₂CH₂CH₂CH₂CH₂ | | OCH₂CH₃ |
| 490 | CH₃ | H | H | OC(CH₃)₃ |
| 491 | CH₃ | phenyl | H | OC(CH₃)₃ |
| 492 | CH₃ | CH₂-indol-3-yl | H | OC(CH₃)₃ |
| 493 | CH₃ | CH₂-(5-hydroxyindol-3-yl) | H | OC(CH₃)₃ |
| 494 | CH₃ | CH₂-(imidazol-4-yl) | H | OC(CH₃)₃ |
| 495 | CH₃ | CH₂-phenyl | H | OC(CH₃)₃ |
| 496 | CH₃ | CH₂-(4-OH-phenyl) | H | OC(CH₃)₃ |
| 497 | CH₃ | CH₂-cyclohexyl | H | OC(CH₃)₃ |
| 498 | CH₃ | CH₃ | H | OC(CH₃)₃ |
| 499 | CH₃ | CH(CH₃)₂ | H | OC(CH₃)₃ |
| 500 | CH₃ | CH₂CH(CH₃)₂ | H | OC(CH₃)₃ |
| 501 | CH₃ | CH(CH₃)CH₂CH₃ | H | OC(CH₃)₃ |
| 502 | CH₃ | (S)—CH(CH₃)CH₂CH₃ | H | OC(CH₃)₃ |
| 503 | CH₃ | (R)—CH(CH₃)CH₂CH₃ | H | OC(CH₃)₃ |
| 504 | CH₃ | CH₂—SH | H | OC(CH₃)₃ |
| 505 | CH₃ | CH₂—CH₂—SH | H | OC(CH₃)₃ |
| 506 | CH₃ | CH₂—SCH₃ | H | OC(CH₃)₃ |
| 507 | CH₃ | CH₂—CH₂—SCH₃ | H | OC(CH₃)₃ |
| 508 | CH₃ | CH₂—CO—NH₂ | H | OC(CH₃)₃ |
| 509 | CH₃ | CH₂—CO—OH | H | OC(CH₃)₃ |
| 510 | CH₃ | CH₂—CO—OCH₃ | H | OC(CH₃)₃ |
| 511 | CH₃ | CH₂—CO—OC₂H₅ | H | OC(CH₃)₃ |
| 512 | CH₃ | CH₂—CO—OCH(CH₃)₂ | H | OC(CH₃)₃ |
| 513 | CH₃ | CH₂—CO—OC(CH₃)₃ | H | OC(CH₃)₃ |
| 514 | CH₃ | CH₂—CH₂—CO—NH₂ | H | OC(CH₃)₃ |
| 515 | CH₃ | CH₂—CH₂—CO—OH | H | OC(CH₃)₃ |
| 516 | CH₃ | CH₂—CH₂—CO—OCH₃ | H | OC(CH₃)₃ |
| 517 | CH₃ | CH₂—CH₂—CO—OC₂H₅ | H | OC(CH₃)₃ |
| 518 | CH₃ | CH₂—CH₂—CO—OCH(CH₃)₂ | H | OC(CH₃)₃ |
| 519 | CH₃ | CH₂—CH₂—CO—OC(CH₃)₃ | H | OC(CH₃)₃ |
| 520 | CH₃ | CH₂—OH | H | OC(CH₃)₃ |
| 521 | CH₃ | CH₂—OCH₃ | H | OC(CH₃)₃ |
| 522 | CH₃ | CH₂—OCH(CH₃)₂ | H | OC(CH₃)₃ |
| 523 | CH₃ | CH₂—OC(CH₃)₃ | H | OC(CH₃)₃ |
| 524 | CH₃ | CHCH₃—OH | H | OC(CH₃)₃ |
| 525 | CH₃ | CHCH₃—OCH₃ | H | OC(CH₃)₃ |
| 526 | CH₃ | CHCH₃—OCH(CH₃)₂ | H | OC(CH₃)₃ |
| 527 | CH₃ | CHCH₃—OC(CH₃)₃ | H | OC(CH₃)₃ |
| 528 | CH₃ | CH₂CH₂ | | OC(CH₃)₃ |
| 529 | CH₃ | CH₂CH₂CH₂ | | OC(CH₃)₃ |
| 530 | CH₃ | CH₂CH₂CH₂CH₂ | | OC(CH₃)₃ |
| 531 | CH₃ | CH₂CH₂CH₂CH₂CH₂ | | OC(CH₃)₃ |
| 532 | CH₃ | H | H | OCH₂CH═CH₂ |
| 533 | CH₃ | phenyl | H | OCH₂CH═CH₂ |
| 534 | CH₃ | CH₂-indol-3-yl | H | OCH₂CH═CH₂ |
| 535 | CH₃ | CH₂-(5-hydroxyindol-3-yl) | H | OCH₂CH═CH₂ |
| 536 | CH₃ | CH₂-(imidazol-4-yl) | H | OCH₂CH═CH₂ |
| 537 | CH₃ | CH₂-phenyl | H | OCH₂CH═CH₂ |
| 538 | CH₃ | CH₂-(4-OH-phenyl) | H | OCH₂CH═CH₂ |
| 539 | CH₃ | CH₂-cyclohexyl | H | OCH₂CH═CH₂ |
| 540 | CH₃ | CH₃ | H | OCH₂CH═CH₂ |
| 541 | CH₃ | CH(CH₃)₂ | H | OCH₂CH═CH₂ |
| 542 | CH₃ | CH₂CH(CH₃)₂ | H | OCH₂CH═CH₂ |
| 543 | CH₃ | CH(CH₃)CH₂CH₃ | H | OCH₂CH═CH₂ |
| 544 | CH₃ | (S)—CH(CH₃)CH₂CH₃ | H | OCH₂CH═CH₂ |
| 545 | CH₃ | (R)—CH(CH₃)CH₂CH₃ | H | OCH₂CH═CH₂ |
| 546 | CH₃ | CH₂—SH | H | OCH₂CH═CH₂ |
| 547 | CH₃ | CH₂—CH₂—SH | H | OCH₂CH═CH₂ |
| 548 | CH₃ | CH₂—SCH₃ | H | OCH₂CH═CH₂ |
| 549 | CH₃ | CH₂—CH₂—SCH₃ | H | OCH₂CH═CH₂ |
| 550 | CH₃ | CH₂—CO—NH₂ | H | OCH₂CH═CH₂ |
| 551 | CH₃ | CH₂—CO—OH | H | OCH₂CH═CH₂ |
| 552 | CH₃ | CH₂—CO—OCH₃ | H | OCH₂CH═CH₂ |
| 553 | CH₃ | CH₂—CO—OC₂H₅ | H | OCH₂CH═CH₂ |
| 554 | CH₃ | CH₂—CO—OCH(CH₃)₂ | H | OCH₂CH═CH₂ |
| 555 | CH₃ | CH₂—CO—OC(CH₃)₃ | H | OCH₂CH═CH₂ |
| 556 | CH₃ | CH₂—CH₂—CO—NH₂ | H | OCH₂CH═CH₂ |
| 557 | CH₃ | CH₂—CH₂—CO—OH | H | OCH₂CH═CH₂ |
| 558 | CH₃ | CH₂—CH₂—CO—OCH₃ | H | OCH₂CH═CH₂ |
| 559 | CH₃ | CH₂—CH₂—CO—OC₂H₅ | H | OCH₂CH═CH₂ |
| 560 | CH₃ | CH₂—CH₂—CO—OCH(CH₃)₂ | H | OCH₂CH═CH₂ |
| 561 | CH₃ | CH₂—CH₂—CO—OC(CH₃)₃ | H | OCH₂CH═CH₂ |
| 562 | CH₃ | CH₂—OH | H | OCH₂CH═CH₂ |
| 563 | CH₃ | CH₂—OCH₃ | H | OCH₂CH═CH₂ |
| 564 | CH₃ | CH₂—OCH(CH₃)₂ | H | OCH₂CH═CH₂ |
| 565 | CH₃ | CH₂—OC(CH₃)₃ | H | OCH₂CH═CH₂ |
| 566 | CH₃ | CHCH₃—OH | H | OCH₂CH═CH₂ |
| 567 | CH₃ | CHCH₃—OCH₃ | H | OCH₂CH═CH₂ |
| 568 | CH₃ | CHCH₃—OCH(CH₃)₂ | H | OCH₂CH═CH₂ |
| 569 | CH₃ | CHCH₃—OC(CH₃)₃ | H | OCH₂CH═CH₂ |
| 570 | CH₃ | CH₂CH₂ | | OCH₂CH═CH₂ |
| 571 | CH₃ | CH₂CH₂CH₂ | | OCH₂CH═CH₂ |
| 572 | CH₃ | CH₂CH₂CH₂CH₂ | | OCH₂CH═CH₂ |
| 573 | CH₃ | CH₂CH₂CH₂CH₂CH₂ | | OCH₂CH═CH₂ |
| 574 | CH₃ | H | H | NHCH₃ |
| 575 | CH₃ | phenyl | H | NHCH₃ |
| 576 | CH₃ | CH₂-indol-3-yl | H | NHCH₃ |
| 577 | CH₃ | CH₂-(5-hydroxyindol-3-yl) | H | NHCH₃ |
| 578 | CH₃ | CH₂-(imidazol-4-yl) | H | NHCH₃ |
| 579 | CH₃ | CH₂-phenyl | H | NHCH₃ |
| 580 | CH₃ | CH₂-(4-OH-phenyl) | H | NHCH₃ |
| 581 | CH₃ | CH₂-cyclohexyl | H | NHCH₃ |
| 582 | CH₃ | CH₃ | H | NHCH₃ |
| 583 | CH₃ | CH(CH₃)₂ | H | NHCH₃ |
| 584 | CH₃ | CH₂CH(CH₃)₂ | H | NHCH₃ |
| 585 | CH₃ | CH(CH₃)CH₂CH₃ | H | NHCH₃ |
| 586 | CH₃ | (S)—CH(CH₃)CH₂CH₃ | H | NHCH₃ |
| 587 | CH₃ | (R)—CH(CH₃)CH₂CH₃ | H | NHCH₃ |
| 588 | CH₃ | CH₂—SH | H | NHCH₃ |
| 589 | CH₃ | CH₂—CH₂—SH | H | NHCH₃ |
| 590 | CH₃ | CH₂—SCH₃ | H | NHCH₃ |
| 591 | CH₃ | CH₂—CH₂—SCH₃ | H | NHCH₃ |
| 592 | CH₃ | CH₂—CO—NH₂ | H | NHCH₃ |
| 593 | CH₃ | CH₂—CO—OH | H | NHCH₃ |
| 594 | CH₃ | CH₂—CO—OCH₃ | H | NHCH₃ |
| 595 | CH₃ | CH₂—CO—OC₂H₅ | H | NHCH₃ |
| 596 | CH₃ | CH₂—CO—OCH(CH₃)₂ | H | NHCH₃ |
| 597 | CH₃ | CH₂—CO—OC(CH₃)₃ | H | NHCH₃ |
| 598 | CH₃ | CH₂—CH₂—CO—NH₂ | H | NHCH₃ |
| 599 | CH₃ | CH₂—CH₂—CO—OH | H | NHCH₃ |
| 600 | CH₃ | CH₂—CH₂—CO—OCH₃ | H | NHCH₃ |
| 601 | CH₃ | CH₂—CH₂—CO—OC₂H₅ | H | NHCH₃ |
| 602 | CH₃ | CH₂—CH₂—CO—OCH(CH₃)₂ | H | NHCH₃ |
| 603 | CH₃ | CH₂—CH₂—CO—OC(CH₃)₃ | H | NHCH₃ |
| 604 | CH₃ | CH₂—OH | H | NHCH₃ |
| 605 | CH₃ | CH₂—OCH₃ | H | NHCH₃ |
| 606 | CH₃ | CH₂—OCH(CH₃)₂ | H | NHCH₃ |
| 607 | CH₃ | CH₂—OC(CH₃)₃ | H | NHCH₃ |
| 608 | CH₃ | CHCH₃—OH | H | NHCH₃ |
| 609 | CH₃ | CHCH₃—OCH₃ | H | NHCH₃ |
| 610 | CH₃ | CHCH₃—OCH(CH₃)₂ | H | NHCH₃ |
| 611 | CH₃ | CHCH₃—OC(CH₃)₃ | H | NHCH₃ |
| 612 | CH₃ | CH₂CH₂ | | NHCH₃ |
| 613 | CH₃ | CH₂CH₂CH₂ | | NHCH₃ |
| 614 | CH₃ | CH₂CH₂CH₂CH₂ | | NHCH₃ |
| 615 | CH₃ | CH₂CH₂CH₂CH₂CH₂ | | NHCH₃ |
| 616 | CH₃ | | H | NHCH₃ |
| 617 | H | H | CH₂CH₂O* | |
| 618 | H | H | CH₂CH₂CH₂O* | |
| 619 | H | H | CH₂CH₂CH₂CH₂O* | |
| 620 | H | H | CH₂CH₂NH* | |
| 621 | H | H | CH₂CH₂CH₂NH* | |
| 622 | H | H | CH₂CH₂CH₂CH₂NH* | |
| 623 | H | H | CH₂CH₂NCH₃* | |
| 624 | H | H | CH₂CH₂CH₂NCH₃* | |
| 625 | H | H | CH₂CH₂CH₂CH₂NCH₃* | |
| 626 | CH₃ | H | CH₂CH₂O* | |
| 627 | CH₃ | H | CH₂CH₂CH₂O* | |
| 628 | CH₃ | H | CH₂CH₂CH₂CH₂O* | |
| 629 | CH₃ | H | CH₂CH₂NH* | |
| 630 | CH₃ | H | CH₂CH₂CH₂NH* | |
| 631 | CH₃ | H | CH₂CH₂CH₂CH₂NH* | |
| 632 | CH₃ | H | CH₂CH₂NCH₃* | |
| 633 | CH₃ | H | CH₂CH₂CH₂NCH₃* | |
| 634 | CH₃ | H | CH₂CH₂CH₂CH₂NCH₃* | |
| 635 | H | H | H | N(CH₃)₂ |
| 636 | H | phenyl | H | N(CH₃)₂ |
| 637 | H | CH₂-indol-3-yl | H | N(CH₃)₂ |
| 638 | H | CH₂-(5-hydroxyindol-3-yl) | H | N(CH₃)₂ |
| 639 | H | CH₂-(imidazol-4-yl) | H | N(CH₃)₂ |
| 640 | H | CH₂-phenyl | H | N(CH₃)₂ |
| 641 | H | CH₂-(4-OH-phenyl) | H | N(CH₃)₂ |
| 642 | H | CH₂-cyclohexyl | H | N(CH₃)₂ |
| 643 | H | CH₃ | H | N(CH₃)₂ |
| 644 | H | CH(CH₃)₂ | H | N(CH₃)₂ |
| 645 | H | CH₂CH(CH₃)₂ | H | N(CH₃)₂ |
| 646 | H | CH(CH₃)CH₂CH₃ | H | N(CH₃)₂ |
| 647 | H | (S)—CH(CH₃)CH₂CH₃ | H | N(CH₃)₂ |
| 648 | H | (R)—CH(CH₃)CH₂CH₃ | H | N(CH₃)₂ |
| 649 | H | CH₂—SH | H | N(CH₃)₂ |
| 650 | H | CH₂—CH₂—SH | H | N(CH₃)₂ |
| 651 | H | CH₂—SCH₃ | H | N(CH₃)₂ |
| 652 | H | CH₂—CH₂—SCH₃ | H | N(CH₃)₂ |
| 653 | H | CH₂—CO—NH₂ | H | N(CH₃)₂ |
| 654 | H | CH₂—CO—OH | H | N(CH₃)₂ |
| 655 | H | CH₂—CO—OCH₃ | H | N(CH₃)₂ |
| 656 | H | CH₂—CO—OC₂H₅ | H | N(CH₃)₂ |
| 657 | H | CH₂—CO—OCH(CH₃)₂ | H | N(CH₃)₂ |
| 658 | H | CH₂—CO—OC(CH₃)₃ | H | N(CH₃)₂ |
| 659 | H | CH₂—CH₂—CO—NH₂ | H | N(CH₃)₂ |
| 660 | H | CH₂—CH₂—CO—OH | H | N(CH₃)₂ |
| 661 | H | CH₂—CH₂—CO—OCH₃ | H | N(CH₃)₂ |
| 662 | H | CH₂—CH₂—CO—OC₂H₅ | H | N(CH₃)₂ |
| 663 | H | CH₂—CH₂—CO—OCH(CH₃)₂ | H | N(CH₃)₂ |
| 664 | H | CH₂—CH₂—CO—OC(CH₃)₃ | H | N(CH₃)₂ |
| 665 | H | CH₂—OH | H | N(CH₃)₂ |
| 666 | H | CH₂—OCH₃ | H | N(CH₃)₂ |
| 667 | H | CH₂—OCH(CH₃)₂ | H | N(CH₃)₂ |
| 668 | H | CH₂—OC(CH₃)₃ | H | N(CH₃)₂ |
| 669 | H | CHCH₃—OH | H | N(CH₃)₂ |
| 670 | H | CHCH₃—OCH₃ | H | N(CH₃)₂ |
| 671 | H | CHCH₃—OCH(CH₃)₂ | H | N(CH₃)₂ |
| 672 | H | CHCH₃—OC(CH₃)₃ | H | N(CH₃)₂ |
| 673 | CH₂CH₂CH₂ | | H | N(CH₃)₂ |
| 674 | CH₂CH(OH)CH₂ | | H | N(CH₃)₂ |
| 675 | CH₂CH₂CH₂CH₂ | | H | N(CH₃)₂ |
| 676 | H | CH₂CH₂ | | N(CH₃)₂ |
| 677 | H | CH₂CH₂CH₂ | | N(CH₃)₂ |
| 678 | H | CH₂CH₂CH₂CH₂ | | N(CH₃)₂ |
| 679 | H | CH₂CH₂CH₂CH₂CH₂ | | N(CH₃)₂ |
| 680 | H | H | H | 1-pyrrolidinyl |
| 681 | H | Phenyl | H | 1-pyrrolidinyl |
| 682 | H | CH₂-Indol-3-yl | H | 1-pyrrolidinyl |
| 683 | H | CH₂-(5-Hydroxyindol-3-yl) | H | 1-pyrrolidinyl |
| 684 | H | CH₂-(Imidazol-4-yl) | H | 1-pyrrolidinyl |
| 685 | H | CH₂-Phenyl | H | 1-pyrrolidinyl |
| 686 | H | CH₂-(4-OH-Phenyl) | H | 1-pyrrolidinyl |
| 687 | H | CH₂-Cyclohexyl | H | 1-pyrrolidinyl |
| 688 | H | CH₃ | H | 1-pyrrolidinyl |
| 689 | H | CH(CH₃)₂ | H | 1-pyrrolidinyl |
| 690 | H | CH₂CH(CH₃)₂ | H | 1-pyrrolidinyl |
| 691 | H | CH(CH₃)CH₂CH₃ | H | 1-pyrrolidinyl |
| 692 | H | (S)—CH(CH₃)CH₂CH₃ | H | 1-pyrrolidinyl |
| 693 | H | (R)—CH(CH₃)CH₂CH₃ | H | 1-pyrrolidinyl |
| 694 | H | CH₂—SH | H | 1-pyrrolidinyl |
| 695 | H | CH₂—CH₂—SH | H | 1-pyrrolidinyl |
| 696 | H | CH₂—SCH₃ | H | 1-pyrrolidinyl |
| 697 | H | CH₂—CH₂—SCH₃ | H | 1-pyrrolidinyl |
| 698 | H | CH₂—CO—NH₂ | H | 1-pyrrolidinyl |
| 699 | H | CH₂—CO—OH | H | 1-pyrrolidinyl |
| 700 | H | CH₂—CO—OCH₃ | H | 1-pyrrolidinyl |
| 701 | H | CH₂—CO—OC₂H₅ | H | 1-pyrrolidinyl |
| 702 | H | CH₂—CO—OCH(CH₃)₂ | H | 1-pyrrolidinyl |
| 703 | H | CH₂—CO—OC(CH₃)₃ | H | 1-pyrrolidinyl |
| 704 | H | CH₂—CH₂—CO—NH₂ | H | 1-pyrrolidinyl |
| 705 | H | CH₂—CH₂—CO—OH | H | 1-pyrrolidinyl |
| 706 | H | CH₂—CH₂—CO—OCH₃ | H | 1-pyrrolidinyl |
| 707 | H | CH₂—CH₂—CO—OC₂H₅ | H | 1-pyrrolidinyl |
| 708 | H | CH₂—CH₂—CO—OCH(CH₃)₂ | H | 1-pyrrolidinyl |
| 709 | H | CH₂—CH₂—CO—OC(CH₃)₃ | H | 1-pyrrolidinyl |
| 710 | H | CH₂—OH | H | 1-pyrrolidinyl |
| 711 | H | CH₂—OCH₃ | H | 1-pyrrolidinyl |
| 712 | H | CH₂—OCH(CH₃)₂ | H | 1-pyrrolidinyl |
| 713 | H | CH₂—OC(CH₃)₃ | H | 1-pyrrolidinyl |
| 714 | H | CHCH₃—OH | H | 1-pyrrolidinyl |
| 715 | H | CHCH₃—OCH₃ | H | 1-pyrrolidinyl |
| 716 | H | CHCH₃—OCH(CH₃)₂ | H | 1-pyrrolidinyl |
| 717 | H | CHCH₃—OC(CH₃)₃ | H | 1-pyrrolidinyl |
| 718 | CH₂CH₂CH₂ | | H | 1-pyrrolidinyl |
| 719 | CH₂CH(OH)CH₂ | | H | 1-pyrrolidinyl |
| 720 | CH₂CH₂CH₂CH₂ | | H | 1-pyrrolidinyl |
| 721 | H | CH₂CH₂ | | 1-pyrrolidinyl |
| 722 | H | CH₂CH₂CH₂ | | 1-pyrrolidinyl |
| 723 | H | CH₂CH₂CH₂CH₂ | | 1-pyrrolidinyl |
| 724 | H | CH₂CH₂CH₂CH₂CH₂ | | 1-pyrrolidinyl |
| 725 | H | H | H | 1-piperidinyl |
| 726 | H | Phenyl | H | 1-piperidinyl |
| 727 | H | CH₂-Indol-3-yl | H | 1-piperidinyl |
| 728 | H | CH₂-(5-Hydroxyindol-3-yl) | H | 1-piperidinyl |
| 729 | H | CH₂-(Imidazol-4-yl) | H | 1-piperidinyl |
| 730 | H | CH₂-Phenyl | H | 1-piperidinyl |
| 731 | H | CH₂-(4-OH-Phenyl) | H | 1-piperidinyl |
| 732 | H | CH₂-Cyclohexyl | H | 1-piperidinyl |
| 733 | H | CH₃ | H | 1-piperidinyl |
| 734 | H | CH(CH₃)₂ | H | 1-piperidinyl |
| 735 | H | CH₂CH(CH₃)₂ | H | 1-piperidinyl |
| 736 | H | CH(CH₃)CH₂CH₃ | H | 1-piperidinyl |
| 737 | H | (S)—CH(CH₃)CH₂CH₃ | H | 1-piperidinyl |
| 738 | H | (R)—CH(CH₃)CH₂CH₃ | H | 1-piperidinyl |
| 739 | H | CH₂—SH | H | 1-piperidinyl |
| 740 | H | CH₂—CH₂—SH | H | 1-piperidinyl |
| 741 | H | CH₂—SCH₃ | H | 1-piperidinyl |
| 742 | H | CH₂—CH₂—SCH₃ | H | 1-piperidinyl |
| 743 | H | CH₂—CO—NH₂ | H | 1-piperidinyl |
| 744 | H | CH₂—CO—OH | H | 1-piperidinyl |
| 745 | H | CH₂—CO—OCH₃ | H | 1-piperidinyl |
| 746 | H | CH₂—CO—OC₂H₅ | H | 1-piperidinyl |
| 747 | H | CH₂—CO—OCH(CH₃)₂ | H | 1-piperidinyl |
| 748 | H | CH₂—CO—OC(CH₃)₃ | H | 1-piperidinyl |
| 749 | H | CH₂—CH₂—CO—NH₂ | H | 1-piperidinyl |
| 750 | H | CH₂—CH₂—CO—OH | H | 1-piperidinyl |
| 751 | H | CH₂—CH₂—CO—OCH₃ | H | 1-piperidinyl |
| 752 | H | CH₂—CH₂—CO—OC₂H₅ | H | 1-piperidinyl |
| 753 | H | CH₂—CH₂—CO—OCH(CH₃)₂ | H | 1-piperidinyl |
| 754 | H | CH₂—CH₂—CO—OC(CH₃)₃ | H | 1-piperidinyl |
| 755 | H | CH₂—OH | H | 1-piperidinyl |
| 756 | H | CH₂—OCH₃ | H | 1-piperidinyl |
| 757 | H | CH₂—OCH(CH₃)₂ | H | 1-piperidinyl |
| 758 | H | CH₂—OC(CH₃)₃ | H | 1-piperidinyl |
| 759 | H | CHCH₃—OH | H | 1-piperidinyl |
| 760 | H | CHCH₃—OCH₃ | H | 1-piperidinyl |
| 761 | H | CHCH₃—OCH(CH₃)₂ | H | 1-piperidinyl |
| 762 | H | CHCH₃—OC(CH₃)₃ | H | 1-piperidinyl |
| 763 | CH₂CH₂CH₂ | | H | 1-piperidinyl |
| 764 | CH₂CH(OH)CH₂ | | H | 1-piperidinyl |
| 765 | CH₂CH₂CH₂CH₂ | | H | 1-piperidinyl |
| 766 | H | CH₂CH₂ | | 1-piperidinyl |
| 767 | H | CH₂CH₂CH₂ | | 1-piperidinyl |
| 768 | H | CH₂CH₂CH₂CH₂ | | 1-piperidinyl |
| 769 | H | CH₂CH₂CH₂CH₂CH₂ | | 1-piperidinyl |
| 770 | H | H | H | morpholin-4-yl |
| 771 | H | phenyl | H | morpholin-4-yl |
| 772 | H | CH₂-indol-3-yl | H | morpholin-4-yl |
| 773 | H | CH₂-(5-hydroxyindol-3-yl) | H | morpholin-4-yl |
| 774 | H | CH₂-(imidazol-4-yl) | H | morpholin-4-yl |
| 775 | H | CH₂-phenyl | H | morpholin-4-yl |
| 776 | H | CH₂-(4-OH-phenyl) | H | morpholin-4-yl |
| 777 | H | CH₂-cyclohexyl | H | morpholin-4-yl |
| 778 | H | CH₃ | H | morpholin-4-yl |
| 779 | H | CH(CH₃)₂ | H | morpholin-4-yl |
| 780 | H | CH₂CH(CH₃)₂ | H | morpholin-4-yl |
| 781 | H | CH(CH₃)CH₂CH₃ | H | morpholin-4-yl |
| 782 | H | (S)—CH(CH₃)CH₂CH₃ | H | morpholin-4-yl |
| 783 | H | (R)—CH(CH₃)CH₂CH₃ | H | morpholin-4-yl |
| 784 | H | CH₂—SH | H | morpholin-4-yl |
| 785 | H | CH₂—CH₂—SH | H | morpholin-4-yl |
| 786 | H | CH₂—SCH₃ | H | morpholin-4-yl |
| 787 | H | CH₂—CH₂—SCH₃ | H | morpholin-4-yl |
| 788 | H | CH₂—CO—NH₂ | H | morpholin-4-yl |
| 789 | H | CH₂—CO—OH | H | morpholin-4-yl |
| 790 | H | CH₂—CO—OCH₃ | H | morpholin-4-yl |
| 791 | H | CH₂—CO—OC₂H₅ | H | morpholin-4-yl |
| 792 | H | CH₂—CO—OCH(CH₃)₂ | H | morpholin-4-yl |
| 793 | H | CH₂—CO—OC(CH₃)₃ | H | morpholin-4-yl |
| 794 | H | CH₂—CH₂—CO—NH₂ | H | morpholin-4-yl |
| 795 | H | CH₂—CH₂—CO—OH | H | morpholin-4-yl |
| 796 | H | CH₂—CH₂—CO—OCH₃ | H | morpholin-4-yl |
| 797 | H | CH₂—CH₂—CO—OC₂H₅ | H | morpholin-4-yl |
| 798 | H | CH₂—CH₂—CO—OCH(CH₃)₂ | H | morpholin-4-yl |
| 799 | H | CH₂—CH₂—CO—OC(CH₃)₃ | H | morpholin-4-yl |
| 800 | H | CH₂—OH | H | morpholin-4-yl |
| 801 | H | CH₂—OCH₃ | H | morpholin-4-yl |
| 802 | H | CH₂—OCH(CH₃)₂ | H | morpholin-4-yl |
| 803 | H | CH₂—OC(CH₃)₃ | H | morpholin-4-yl |
| 804 | H | CHCH₃—OH | H | morpholin-4-yl |
| 805 | H | CHCH₃—OCH₃ | H | morpholin-4-yl |
| 806 | H | CHCH₃—OCH(CH₃)₂ | H | morpholin-4-yl |
| 807 | H | CHCH₃—OC(CH₃)₃ | H | morpholin-4-yl |

| | | | |
|---|---|---|---|
| 808 | CH₂CH₂CH₂ | H | morpholin-4-yl |
| 809 | CH₂CH(OH)CH₂ | H | morpholin-4-yl |
| 810 | CH₂CH₂CH₂CH₂ | H | morpholin-4-yl |

| | | | |
|---|---|---|---|
| 811 | H | CH₂CH₂ | morpholin-4-yl |
| 812 | H | CH₂CH₂CH₂ | morpholin-4-yl |
| 813 | H | CH₂CH₂CH₂CH₂ | morpholin-4-yl |
| 814 | H | CH₂CH₂CH₂CH₂CH₂ | morpholin-4-yl |

*the heteroatom is attached to the carbon atom of the carbonyl group

Other examples of preferred compounds of the formula I according to the invention are the enantiomers of the formulae I-L′ and I-D′ and the racemate of the formula I-R′ in which R⁷ is hydrogen or methyl, X and Lₘ have the meanings given in tables 1 to 60 and the variables R¹, R², R³ and Y in each case together have the meaning given in one of rows 1 to 814 of table A:

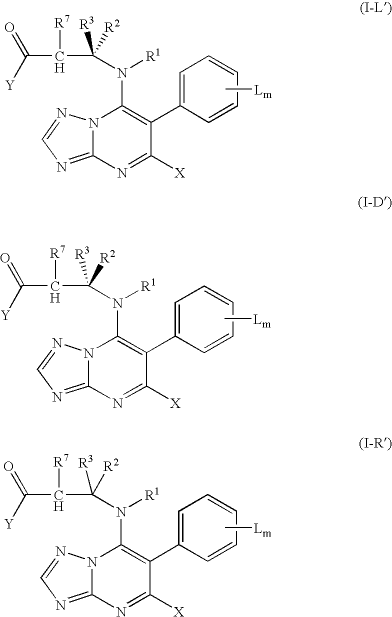

Other examples of preferred compounds of the formula I according to the invention are the enantiomers of the formula I-L″ and I-D″ and the racemate of the formula I-R″ in which Lₘ and X have the meanings given in tables 1 to 60 and the variable Y in each case together has the meaning given in one of rows 1 to 16 of table A′:

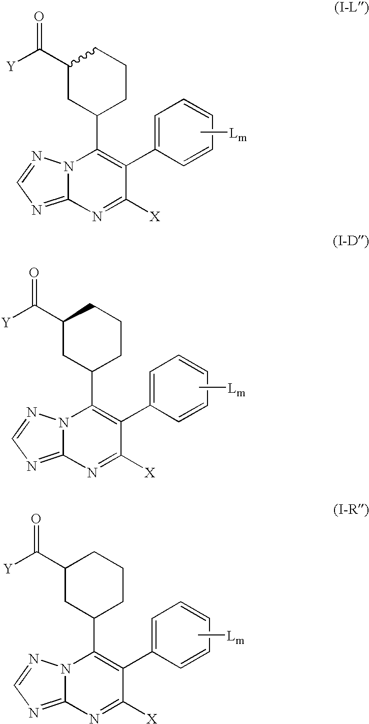

TABLE A

| No. | Y |
|---|---|
| 1 | OH |
| 2 | OCH₃ |
| 3 | OCH₂CH₃ |
| 4 | OCH₂CH₂CH₃ |
| 5 | OCH═CH—CH₃ |
| 6 | OCH(CH₃)₂ |
| 7 | OCH₂CH₂OCH₃ |
| 8 | OC(CH₃)₃ |
| 9 | OCH₂C₆H₅ |
| 10 | NH₂ |
| 11 | NHCH₃ |
| 12 | NHCH₂CH₃ |
| 13 | N(CH₃)2 |
| 14 | 1-pyrrolidinyl |
| 15 | 1-piperidinyl |
| 16 | morpholin-4-yl |

The compounds according to the invention can be obtained by different routes. The compounds I in which X is halogen and W is oxygen (compounds I.A) are generally prepared by reacting 5,7-dihalotriazolopyrimidines of the formula II with aminoocarboxylic acid derivatives of the formula II, according to the method shown in scheme 1:

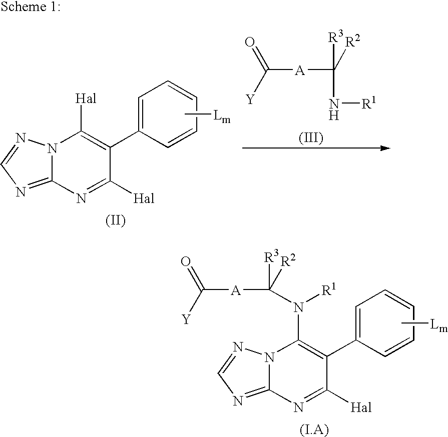

In Scheme 1, R¹-R³, L, m and Y are as defined above. Hal is halogen, in particular chlorine. The reaction of II with aminocarboxylic acid derivative is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO 98/46608; WO 02/48151].

The use of a base such as a tertiary amine, for example triethylamine, or an inorganic base, such as potassium carbonate, is preferred; it is also possible for excess aminocarboxylic acid of the formula III to serve as base.

The amino acid derivatives of the formula III are known, and most of them are commercially available or can be prepared by known methods for preparing and derivativatizing amino acids. 5,7-Dihalotriazolopyrimidines of the formula II are known from the prior art cited at the outset or can be prepared analogously to methods described therein.

Compounds of the formula I in which X is cyano or C₁-C₄-alkoxy (formula I.B) can be prepared advantageously from compounds I.A by the method shown in scheme 2.

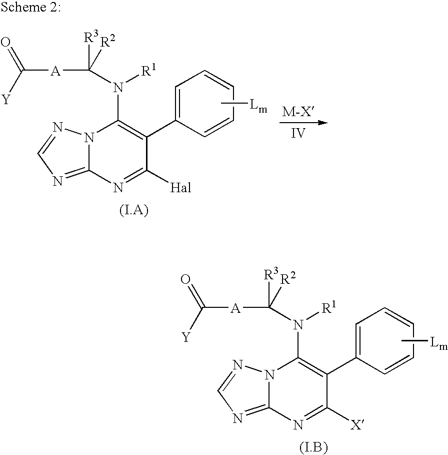

In scheme 2, R¹-R³, Hal, L, m and Y are as defined above. X′ is cyanide, C₁-C₄-alkoxide or C₁-C₄-haloalkoxide. The reaction is advantageously carried out in the presence of an inert solvent. The cation M in the formula IV is of little importance; for practical reasons, ammonium, tetraalkylammonium or alkali metal or alkaline earth metal salts are usually preferred. The reaction temperature is usually from 0 to 120° C., preferably from 10 to 40° C. [cf. J. Heterocycl. Chem. 12 (1975), 861-863]. Suitable solvents include ethers, such as dioxane, diethyl ether and, preferably, tetrahydrofuran, halogenated hydrocarbons, such as dichloromethane, and aromatic hydrocarbons, such as toluene.

Compounds I in which X is C₁-C₄-alkyl (formula I.C) can be prepared advantageously from starting materials of the formula I.A by the routes outlined below.

Compounds of the formula I.C in which X″ is C₁-C₄-alkyl can be obtained, for example, by coupling 5-halotriazolopyrimidines of the formula I.A with organometallic reagents of the formula V (see scheme 3). In one embodiment of this process, the reaction is carried out with transition metal catalysis, for example in the presence of catalytic amounts of Ni or Pd compounds.

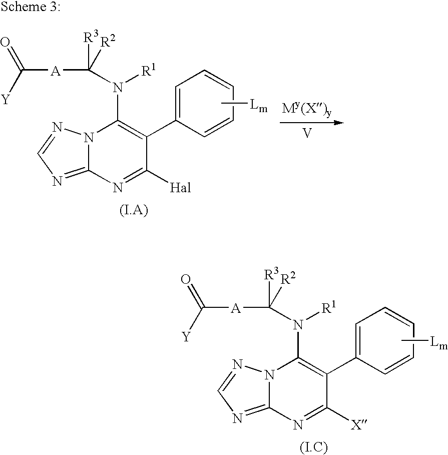

In the formulae I.C and V, X″ is C₁-C₄-alkyl and M is a metal ion of valency Y, such as, for example, B, Zn or Sn. This reaction can be carried out, for example, analogously to the following methods: J. Chem. Soc., Perkin Trans. 1, (1994), 1187, ibid. 1 (1996), 2345; WO 99/41255; Aust. J. Chem. 43 (1990), 733; J. Org. Chem. 43 (1978), 358; J. Chem. Soc., Chem. Commun. (1979), 866; Tetrahedron Lett. 34 (1993), 8267; ibid. 33 (1992), 413.

Compounds of the formula I in which X is C₁-C₄-alkyl or C₁-C₄-haloalkyl (formula I.C) can also be prepared advantageously by the following synthesis route, shown in scheme 4:

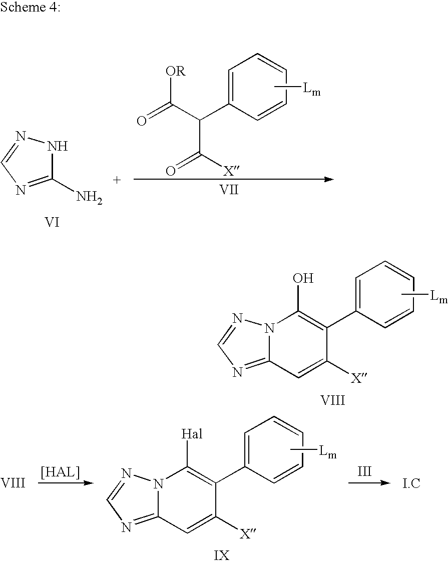

In scheme 4, R¹-R³, L, m and Y are as defined above. Hal is, in particular, chlorine or bromine, X′ is C₁-C₄-alkyl or C₁-C₄-haloalkyl and R is C₁-C₄-alkyl, in particular methyl or ethyl.

In a first step, by methods known per se, by reacting 5-aminotriazole VI with the keto ester VII, a 5-alkyl-7-hydroxy-6-phenyltriazolopyrimidine VIII is prepared [cf. Chem. Pharm. Bull. 9 (1961), 801]. The 5-aminotriazole VI used is commercially available. The starting materials VII are advantageously prepared under the conditions known from EP-A 10 02 788.

The 5-alkyl-7-hydroxy-6-phenyltriazolopyrimidines VIII obtained in this manner are, in a second step, reacted with halogenating agents [HAL] to give 7-halotriazolopyrimidines of the formula IX. Preferred halogenating agents are chlorinating or brominating agents, such as phosphorus oxybromide, phosphorus oxychloride, thionyl chloride, thionyl bromide or sulfuryl chloride. The reaction can be carried out neat or in the presence of a solvent. Customary reaction temperatures are from 0 to 150° C. or, preferably, from 80 to 125° C.

The reaction of the 7-halotriazolopyrimidine 1× with the aminocarboxylic acid derivative of the formula III is advantageously carried out at from 0° C. to 70° C., in particular from 10° C. to 35° C. The reaction is preferably carried out in the presence of an inert solvent, such as an ether, for example dioxane, diethyl ether or, in particular, tetrahydrofuran, a halogenated hydrocarbon, such as dichloromethane, an aromatic hydrocarbon, such as, for example, toluene, xylenes, etc. [cf. WO 98/46608].

Preference is given to using a base, such as a tertiary amine, for example triethylamine, or an inorganic base, such as potassium carbonate; it is also possible for excess aminocarboxylic acid derivative of the formula III to serve as base.

Alternatively, compounds of the formula I.C can also be prepared by reacting compounds I.A with dialkyl malonates of the formula X, followed by decarboxylation, according to the method shown in scheme 5 [cf. U.S. Pat. No. 5,994,360].

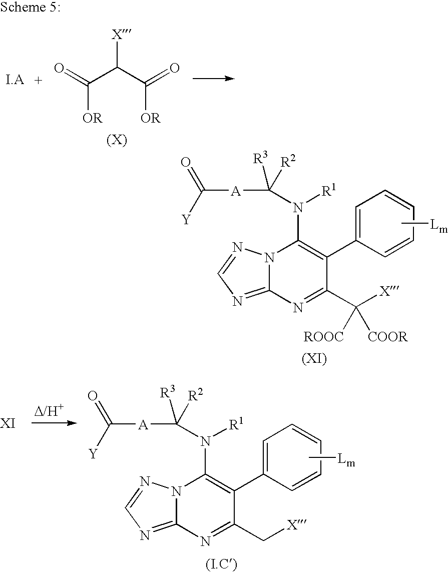

In scheme 5, R¹-R³, L, m and Y are as defined above. X′″ is hydrogen, C₁-C₃-alkyl or C₁-C₃-haloalkyl and R is C₁-C₄-alkyl.

In a first step, the compound I.A is reacted with a dialkyl malonate of the formula X, preferably in the presence of a base, or with the salt of X. This gives the compound XI. The reaction can be carried out analogously to the process described in U.S. Pat. No. 5,994,360. The malonates X are known from the literature [J. Am. Chem. Soc. 64 (1942), 2714; J. Org. Chem. 39 (1974), 2172; Helv. Chim. Acta 61 (1978), 1565], or they can be prepared in accordance with the literature cited.

The subsequent hydrolysis of the ester XI is carried out under generally customary conditions [cf. U.S. Pat. No. 5,994,360]. Depending on the various structural elements, alkaline or acidic hydrolysis of the compounds XI may be advantageous. Under the conditions of ester hydrolysis, there may already be complete or partial decarboxylation to I.C′. The decarboxylation is usually carried out at temperatures of from 20° C. to 180° C., preferably from 50° C. to 120° C., in an inert solvent, if appropriate in the presence of an acid. Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The reaction mixtures obtained by the methods shown in schemes 1 to 5 are worked up in a customary manner, for example by mixing with water, separating the phases and, if appropriate, chromatographically purifying the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification may also be by recrystallization or digestion.

If individual compounds I cannot be obtained by the routes described above, they can be prepared by derivatization of other compounds I.

If the synthesis yields mixtures of isomers, a separation is generally not necessarily required, however, since in some cases the individual isomers can be interconverted during work-up for use or during use (for example under the action of light, acids or bases). Such conversions may also take place after use, for example in the case of treatment of plants in the treated plant, or in the harmful fungus to be controlled.

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

Examples of formulations include products for dilution with water, for example,

A Water-Soluble Concentrates (SL)

10 parts by weight of a compound according to the invention are dissolved in water or in a water-soluble solvent. As an alternative, wetters or other auxiliaries are added.

The active compound dissolves upon dilution with water;

B Dispersible Concentrates (DC)

20 parts by weight of a compound according to the invention are dissolved in cyclohexanone with addition of a dispersant, for example polyvinylpyrrolidone. Dilution with water gives a dispersion;

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion;

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion;

E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound;

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound;

G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound;

and products to be applied undiluted, for example,

H Dustable Powders (DP)

5 parts by weight of a compound according to the invention are ground finely and mixed intimately with 95% of finely divided kaolin. This gives a dustable product;

I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted;

J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume method (ULV), by which it is possible to apply formulations comprising over 95% by weight of active compound, or even to apply the active compound without additives.

Various types of oils, wetting agents, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them, in the use form as fungicides, with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyrodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, toloclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to obtain further compounds by appropriate modification of the starting materials. The compounds thus obtained are listed in the tables below, together with physical data.

### Example 1

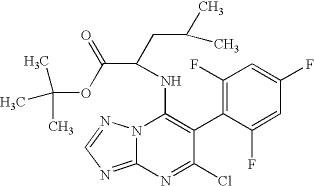

At room temperature, 32,57 μl (0.235 mmol) of triethylamine were added to a mixture of 75 mg (0.235 mmol) of 5,7-dichloro-6-(2,4,6-trifluorophenyl)-[1,2,4]triazolo-[1,5-a]pyrimidine and 44 mg (0.235 mmol) of tert-butyl 2-amino-4-methylpentanoate in 2 ml of dichloromethane. The reaction mixture was stirred at room temperature overnight. The mixture was then extracted twice with in each case 5 ml of 5% strength sodium chloride solution. The organic phase was separated off, dried and concentrated under reduced pressure, which gave the title compound in a yield of >90%.

The compounds of the formula Ia (compounds I, X is chlorine and A is a chemical bond, and Ib (compounds I, X is chlorine and A is a group CHR⁷) listed in tables B and C below were prepared using the procedure given for example 1.

All products were characterized by combined HPLC/mass spectrometry. An analytical RP-18 column (Chromolith Speed ROD from Merck KGaA, Germany), which was operated at 40° C., was used for HPLC. The mobile phase used was acetonitrile with 0.1% by volume of trifluoroacetic acid and a 0.1% by volume of trifluoroacetic acid/water mixture (over a period of 5 min, the ratio trifluoroacetic acid/water was changed from 5:95 to 95:5). Mass spectrometry was carried out using a quadrupole mass spectrometer with electrospray ionization at 80V in the positive mode.

TABLE B

| | |
|---|---|
| | (Ia) |
| 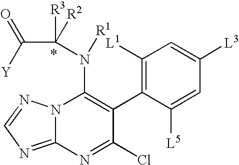 | |

| Ex. | R¹ | R² | R³ | Y | L¹ | L³ | L⁵ | *¹ | min²/m/z³ |
|---|---|---|---|---|---|---|---|---|---|
| 1 | H | CH₂CH(CH₃)₂ | H | OC(CH₃)₃ | F | F | F | rac | 4.08/470.10 |
| 2 | H | H | H | OC(CH₃)₃ | F | F | F | — | 3.37/414.00 |
| 3 | H | (S) CH(CH₃)CH₂CH₃⁴ | H | OCH₃ | F | F | F | S | 3.61/428.00 |
| 4 | H | CH(CH₃)₂ | H | OCH₃ | F | F | F | S | 3.41/428.00 |
| 5 | H | CH₃ | H | OCH₃ | F | F | F | S | 2.96/386.00 |
| 6 | H | CH₂CH(CH₃)₂ | H | OCH₃ | F | F | F | S | 3.56/428.00 |
| 7 | H | CH₂-indol-3-yl | H | OCH₃ | F | F | F | S | 2.46/501.00 |
| 8 | H | CH₂-phenyl | H | OCH₃ | F | F | F | S | 3.54/462.00 |
| 9 | H | CH(CH₃)₂ | H | OC(CH₃)₃ | F | F | F | S | 3.86/456.00 |
| 10 | H | H | H | OCH₂CH₃ | F | F | F | — | 2.87/386.00 |
| 11 | H | CH₂—CH₂—SCH₃ | H | OCH₃ | F | F | F | rac | 3.17/446.00 |
| 12 | H | CH₂—CH₂—CO—OCH₃ | H | OCH₃ | F | F | F | S | 2.95/458.00 |
| 13 | CH₃ | H | H | OCH₂CH₃ | F | F | F | — | 3.08/400.00 |
| 14 | CH₃ | H | H | NHCH₃ | F | F | F | — | 2.40/385.00 |
| 15 | H | (S) CH(CH₃)CH₂CH₃⁴ | H | OCH₂CH═CH₂ | F | F | F | S | 3.74/454.00 |
| 16 | H | CH₂-indol-3-yl | H | OCH₂CH₃ | F | F | F | S | 3.62/515.00 |
| 17 | H | CH₂-phenyl | H | OCH₂CH₃ | F | F | F | S | 3.69/476.00 |
| 18 | H | CH₃ | H | OC(CH₃)₃ | F | F | F | rac | 3.60/428.00 |
| 19 | H | CH₂—CH₂—SCH₃ | H | OC(CH₃)₃ | F | F | F | rac | 3.78/488.00 |
| 20 | H | CH(CH₃)₂ | H | OC(CH₃)₃ | F | F | F | S | 3.98/456.10 |
| 21 | H | phenyl | H | OCH₃ | F | F | F | rac | 3.49/448.00 |
| 22 | H | CH₂—CH₂—SCH₃ | H | OCH₃ | F | F | F | rac | 3.25/446.00 |
| 23 | H | CH₂CH₂ | | OCH₃ | F | F | F | rac | 3.07/412,00 |
| 24 | H | CH₂—CH₂—CO—OC(CH₃)₃ | H | OCH₃ | F | F | F | S | 3.54/486.00 |
| 25 | H | CH₂-cyclohexyl | H | OCH₃ | F | F | F | S | 3.84/486.00 |
| 26 | H | CH₂—CH₂—SCH₃ | H | OCH₂CH═CH₂ | F | F | F | S | 3.41/472.00 |
| 27 | H | (S) CH(CH₃)CH₂CH₃⁴ | H | OCH₂-Phenyl | F | F | F | S | 3.97/504.10 |
| 28 | H | CH(CH₃)₂ | H | OC(CH₃)₃ | F | F | F | S | 3.85/456.00 |
| 29 | H | CH₂—OC(CH₃)₃ | H | OCH₃ | F | F | F | S | 3.51/458.00 |
| 30 | H | CH(CH₃)₂ | H | OCH₂CH₃ | F | F | F | S | 3.61/441 |
| 31 | CH₂CH₂CH₂CH₂ | | H | OCH₃ | F | F | H | rac | — |
| 32 | H | H | CH₂CH₂CH₂CH₂NH⁵ | | F | F | F | rac | 2.74/410 |
| 33 | H | H | H | OH | F | F | F | — | 2.35/357 |
| 34 | H | CH₂CH₂CH₂CH₂ | | OCH₂CH₃ | F | F | F | — | 3.66/454 |
| 35 | CH₂C₆H₅ | H | H | OCH₂CH₃ | F | F | F | — | 3.72/475 |
| 36 | H | CH₂CH(CH₃)₂ | H | OCH₂CH₂CH₃ | F | F | F | S | 3.90/455 |
| 37 | H | CH(CH₃)₂ | H | OCH₂CH₃ | F | F | F | S | 3.53/427 |
| 38 | CH₃ | CH(CH₃)₂ | H | OCH₂CH₂CH₃ | F | F | F | S | 3.22/469 |
| 39 | H | (S) CH(CH₃)CH₂CH₃ | H | OCH₂CH₂CH₃ | F | F | F | S | 3.91/455 |
| 40 | H | CH(CH₃)₂ | H | OCH₂CH₂CH₃ | F | F | F | R | 3.78/441 |
| 41 | H | CH₂CH₃ | H | OCH₃ | F | F | F | rac | 3.14/399 |
| 42 | H | CH(CH₃)₂ | H | OCH₃ | F | H | Cl | S | 3.38/411 |
| 43 | H | CH₂CH(CH₃)₂ | H | OCH₃ | F | H | Cl | S | 3.53/425 |
| 44 | H | (S) CH(CH₃)CH₂CH₃⁴ | H | OCH₃ | F | H | Cl | S | 3.61/425 |
| 45 | H | CH(CH₃)₂ | H | OCH₃ | Cl | OCH₃ | H | S | 3.42/423 |
| 46 | H | CH₂CH(CH₃)₂ | H | OCH₃ | Cl | OCH₃ | H | S | 3.57/437 |
| 47 | H | (S) CH(CH₃)CH₂CH₃⁴ | H | OCH₃ | Cl | OCH₃ | H | S | 3.64/437 |
| 48 | H | CH(CH₃) | H | OH | Cl | OCH₃ | H | S | 3.42/423 |
| 49 | H | CH₂CH(CH₃)₂ | H | OCH₂CH₃ | F | F | F | R | 3.63/441 |
| 50 | H | CH₂CH(CH₃)₂ | H | OCH₂CH₂CH₃ | F | F | F | R | 3.81/455 |
| 51 | H | CH(CH₃)₂ | H | OCH₂CH₂CH₃ | F | F | F | S | 3.74/441 |
| 52 | H | CH(CH₃)₂ | H | OCH₃ | F | F | F | R | 3.33/413 |
| 53 | H | CH₂CH(CH₃)₂ | H | OCH₃ | F | F | F | R | 3.44/427 |
| 54 | H | C(CH₃)₃ | H | NHCH₃ | F | F | F | rac | 2.95/426 |
| 55 | H | (S) CH(CH₃)CH₂CH₃ | H | OCH₂CH₃ | F | F | F | S | 3.70/441 |
| 56 | CH₂CH₂CH₂CH₂ | | H | OCH₃ | F | F | F | rac | 3.53/424 |
| 57 | H | H | | CH₂CH₂O⁵ | F | F | F | rac | 2.64/382 |
| 58 | CH₃ | CH(CH₃)₂ | H | OCH₃ | F | F | F | S | 3.70/441 |
| 59 | CH₃ | (S) CH(CH₃)CH₂CH₃ | H | OCH₃ | F | F | F | S | 3.70/441 |
| 60 | CH₃ | CH(CH₃)₂ | H | OCH₃ | F | F | F | R | 3.61/427 |
| 61 | H | C(CH₃)₃ | H | OCH₃ | F | F | F | rac | 3.56/427 |
| 62 | H | CF₃ | H | H | F | F | F | rac | 2.85/425 |

¹configuration at the α-carbon atom
²HPLC retention time in minutes
³m/z of the [M + H]⁺ peak
⁴configuration of the chiral carbon atom in the side chain R²
⁵heteroatom attached to the carbonyl group

The compounds of the formula Ia′ (compounds I where Lₘ is 2,4,6-trifluoro, X is chlorine and A is a CHR⁷ group) listed in table C below were prepared by the procedure given for example 1.

TABLE C

| | |
|---|---|
| | (Ia′) |
| 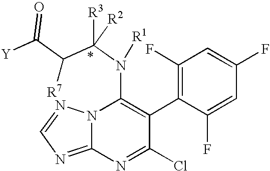 | |

| Ex. | R¹ | R⁷ | R² | R³ | Y | *¹ | min²/m/z³ |
|---|---|---|---|---|---|---|---|
| 63 | CH₂CH₂CH₂ | | H | H | OCH₂CH₃ | rac | 3.49 |
| 64 | H | H | CH₃ | H | OCH₃ | — | 2.79/386 |
| 65 | H | H | H | H | OCH₂CH₃ | rac | 3.21/414 |
| 66 | H | H | H | H | OC(CH₃)₃ | — | 3.48/428 |
| 67 | H | H | phenyl | H | OCH₃ | rac | 3.50/448 |
| 68 | H | H | 4-isopropylphenyl | H | OCH₃ | rac | 3.95/504 |
| 69 | H | H | 4-fluorophenyl | H | OCH₃ | rac | 3.45/480 |
| 70 | H | H | 4-methylphenyl | H | OCH₃ | rac | 3.60/476 |
| 71 | H | H | 2-naphthyl | H | OCH₃ | rac | 3.69/512 |

¹configuration at the α-carbon atom
²HPLC retention time in minutes
³m/z of the [M + H]⁺ peak

### Example 72

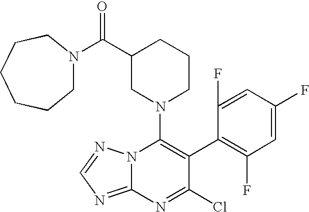

Retention time in HPLC analysis in minutes: 3.42.

m/z: 492 [M+H]⁺

The active compounds were prepared as a stock solution comprising 0.25% by weight of active compound in acetone or DMSO. 1% by weight of the emulsifier Uniperol® EL (wetting agent having emulsifying and dispersing action based on ethoxylated alkylphenols) was added to this solution, and the mixture was diluted with water to the desired concentration.

### Use Example 1—Activity Against Early Blight Caused by Alternaria solani

Leaves of tomato plants of the cultivar “golden princess” were sprayed to runoff point with an aqueous suspension having the concentration of active compound stated below. The next day, the treated plants were infected with a spore suspension of Alternaria solani in a 2% aqueous biomalt solution having a density of 0.17×10⁶ spores/ml. The test plants were then placed in a water-vapor-saturated chamber at temperatures of from 20 to 22° C. After 5 days, the disease on the untreated, but infected plants had developed to such an extent that the infection could be determined visually.

In this test, the plants treated with 250 ppm of the active compounds from examples 3, 4, 37, 42, 52, 56, 13, 30, 43, 45, 46, 47 showed no or only little infection of up to at most 15%, whereas the untreated plants were 90% affected.

### Use Example 2—Activity Against Gray Mold on Bell Pepper Leaves Caused by Botrytis cinerea, Protective Application

Bell pepper leaves of the cultivar “Neusiedler Ideal Elite” were, after 2 to 3 leaves were well-developed, sprayed to runoff point with an aqueous suspension having the concentration of active compound stated below. The next day, the treated plants were inoculated with an aqueous spore suspension of Botrytis cinerea in a 2% aqueous biomalt solution having a density of 0.17×10⁶ spores/ml. The plants were then placed in a climatized chamber at temperatures between 22 and 24° C. and high atmospheric humidity. After 5 days, the extent of the fungal infection was determined visually by the infected leaf area.

In this test, the plants treated with 250 ppm of the active compounds from examples 3, 4, 6, 11, 15, 25, 26, 30, 33, 35, 37, 39, 40, 41, 42, 43, 44, 45, 46, 47, 51, 52, 54, 55, 56, 60, 61 showed no or only very little infection, i.e. less than 10%, whereas the untreated plants were at least 80% infected.

### Use Example 3—Curative Activity Against Brown Rust of Wheat Caused by Puccinia recondita

The active compounds were prepared as a stock solution by mixing 25 mg of active compound with a mixture of acetone and/or DMSO and the emulsifier Uniperol® EL (wetting agent having an emulsifying and dispersing action based on ethoxylated alkylphenols) in a volume ratio of solvent/emulsifier of 99:1 to give a total volume of 10 ml, and the mixture was then diluted to 100 ml with water. This stock solution was diluted with the solvent/emulsifier/water mixture described to give the concentration of active compounds stated below.

Leaves of potted wheat seedlings of the cultivar “Kanzler” were inoculated with a spore suspension of brown rust (Puccinia recondita). The pots were then placed into a chamber with high atmospheric humidity (90 to 95%) and at 20-22° C. for 24 hours. During this time, the spores germinated and the germ tubes penetrated into the leaf tissue. The next day, the infected plants were sprayed to runoff point with an aqueous suspension having the concentration of active compounds stated below. The suspension or emulsion was prepared as described above. After the spray coating had dried on, the test plants were cultivated in a greenhouse at temperatures between 20 and 22° C. and at 65 to 70% relative atmospheric humidity for 7 days. The extent of the rust fungus development on the leaves was then determined.

In this test, the plants treated with 250 ppm of the active compounds from examples 60, 61 showed no infection, whereas the untreated plants were 80% infected.

1. A substituted triazolopyrimidine of the formula I 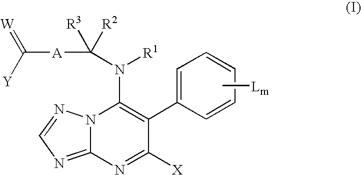
in which:
X is halogen, cyano, C₁-C₄-alkyl, C₁-C₄-haloalkyl, C₁-C₄-alkoxy or C₁-C₂-haloalkoxy;
W is oxygen or sulfur;
Y is O—R⁴ or a group NR⁵R⁶;
A is a chemical bond or a group CR⁷R⁸;
the radicals L independently of one another are halogen, C₁-C₆-alkyl, C₂-C₆-alkenyl, C₁-C₆-haloalkyl, C₁-C₆-alkoxy, nitro, amino, NHR, NR₂, cyano, S(═O)ₙA¹ or C(═O)A², in which
R independently of one another are C₁-C₈-alkyl or C₁-C₈-alkylcarbonyl;
A¹ is hydrogen, hydroxyl, C₁-C₈-alkyl, NH₂, C₁-C₈-alkylamino or di(C₁-C₈-alkyl)amino;
n is 0, 1 or 2;
A² is C₂-C₈-alkenyl, C₁-C₈-alkoxy, C₁-C₆-haloalkoxy, hydrogen, hydroxyl, C₁-C₈-alkyl, NH₂, C₁-C₈-alkylamino or di-(C₁-C₈-alkyl)amino;
m is 0 or 1, 2, 3, 4 or 5;
R¹ is hydrogen, C₁-C₄-alkyl, formyl, C₁-C₄-alkylcarbonyl or C₁-C₄-alkoxycarbonyl or together with R² is C₃-C₆-alkylene where 1 carbon atom may be replaced by an oxygen atom or a sulfur atom and which may optionally carry 1, 2, 3 or 4 C₁-C₄-alkyl groups and/or 1 or 2 radicals R^{a};
R^{a}: is halogen, OH, C₁-C₄-alkoxy or C₁-C₄-alkoxycarbonyl;
R² is hydrogen, C₁-C₆-alkyl which may have a radical R^{b}, C₁-C₄-haloalkyl, C₁-C₄-alkoxy, C₃-C₆-cycloalkyl or phenyl which optionally carries 1, 2 or 3 radicals R^{c};
R^{b}: is OR⁹, SR¹⁰, NR¹¹R¹², COOR¹³, CONR¹⁴R¹⁵, NHC(═NR¹⁶)NR¹⁴R¹⁵, phenyl which optionally carries 1, 2 or 3 radicals R^{c}, 5- or 6-membered heteroaryl which has 1 nitrogen atom and if appropriate 1 or 2 further heteroatoms selected from the group consisting of O, S and N as ring members and which optionally carries 1 or 2 radicals R^{b} or can have a fused-on phenyl ring which for its part can have 1 or 2 radicals R^{b}, or is C₃-C₆-cycloalkyl;
R^{c}: is halogen, C₁-C₄-alkyl, C₁-C₄-haloalkyl, OH, C₁-C₄-alkoxy or C₁-C₄-alkoxycarbonyl;
R³ is hydrogen, C₁-C₄-alkyl or C₁-C₄-alkoxy or together with R² is C₂-C₆-alkylene where 1 carbon atom may be replaced by an oxygen atom or a sulfur atom and which may optionally carry 1, 2, 3 or 4 C₁-C₄-alkyl groups and/or 1 or 2 radicals R^{a};
R⁴ is hydrogen, C₁-C₈-alkyl, hydroxy-C₁-C₄-alkyl, C₁-C₄-alkoxy-C₁-C₄-alkyl, hydroxy-C₁-C₄-alkoxy-C₁-C₄-alkyl, C₁-C₈-haloalkyl, C₂-C₈-alkenyl, C₂-C₈-haloalkenyl, C₃-C₆-cycloalkyl, C₃-C₆-cycloalkyl-C₁-C₄-alkyl, C₃-C₆-halocycloalkyl, C₃-C₈-cycloalkenyl, phenyl, phenyl-C₁-C₄-alkyl, where phenyl in the two last-mentioned radicals may have 1, 2 or 3 of the substituents R^{d} below:
R^{d}: is halogen, cyano, nitro, hydroxyl, mercapto, amino, carboxyl, aminocarbonyl, aminothiocarbonyl, C₁-C₄-alkyl, C₁-C₄-haloalkyl, C₁-C₄-alkoxy, C₁-C₄-haloalkoxy, C₁-C₄-alkylthio, C₁-C₄-alkylamino, C₁-C₄-dialkylamino, C₁-C₄-alkylcarbonyl, C₁-C₄-alkylsulfonyl, C₁-C₄-alkylsulfinyl, C₁-C₄-alkoxycarbonyl, C₁-C₄-alkylcarbonyloxy, C₁-C₄-alkylaminocarbonyl, C₁-C₄-dialkylaminocarbonyl,
or R⁴ together with one of the radicals R¹, R², R³ or R⁷ is C₂-C₆-alkylene where 1 carbon atom may be replaced by an oxygen atom or a sulfur atom and which may optionally carry 1, 2, 3 or 4 radicals selected from the group consisting of halogen and C₁-C₄-alkyl and/or 1 or 2 radicals R^{a};
R⁵, R⁶ independently of one another are hydrogen, C₁-C₈-alkyl, C₃-C₆-cycloalkyl, C₃-C₆-cycloalkyl-C₁-C₄-alkyl, or together with the nitrogen atom to which they are attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups;
or one of the radicals R⁵ or R⁶ together with one of the radicals R¹, R², R³ or R⁷ is C₂-C₆-alkylene where 1 carbon atom may be replaced by an oxygen atom or a sulfur atom and which may optionally carry 1, 2, 3 or 4 radicals selected from the group consisting of halogen and C₁-C₄-alkyl and/or 1 or 2 radicals R^{a};
R⁷, R⁸ independently of one another are hydrogen, C₁-C₄-alkyl or C₁-C₄-alkoxy or one of the radicals R⁷ or R⁸ together with one of the radicals R¹ or R² is C₂-C₆-alkylene where 1 carbon atom may be replaced an oxygen atom or a sulfur atom and which may optionally carry 1, 2, 3 or 4 C₁-C₄-alkyl groups and/or 1 or 2 radicals R^{a};
R⁹ is hydrogen, C₁-C₈-alkyl, formyl or C₁-C₈-alkylcarbonyl;
R¹⁰ is hydrogen or C₁-C₄-alkyl;
R¹¹, R¹² independently of one another are hydrogen, C₁-C₈-alkyl, C₃-C₆-cycloalkyl, C₃-C₆-cycloalkyl-C₁-C₄-alkyl, or together with the nitrogen atom to which they are attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups, where one of the radicals R¹¹, R¹² may also be formyl, C₁-C₈-alkylcarbonyl or C₁-C₈-alkylthiocarbonyl;
R¹³ is hydrogen, C₁-C₈-alkyl, hydroxy-C₁-C₄-alkyl, C₁-C₄-alkoxy-C₁-C₄-alkyl, hydroxy-C₁-C₄-alkoxy-C₁-C₄-alkyl, C₁-C₈-haloalkyl, C₂-C₈-alkenyl, C₂-C₈-haloalkenyl, C₃-C₆-cycloalkyl, C₃-C₆-cycloalkyl-C₁-C₄-alkyl, C₃-C₆-halocycloalkyl, C₃-C₈-cycloalkenyl, phenyl, phenyl-C₁-C₄-alkyl, where phenyl in the two last-mentioned radicals may have 1, 2 or 3 of the abovementioned substituents R^{d};
R¹⁴, R¹⁵ independently of one another are hydrogen, C₁-C₈-alkyl, C₃-C₆-cycloalkyl, C₃-C₆-cycloalkyl-C₁-C₄-alkyl, or together with the nitrogen atom to which are they attached form a saturated 5-, 6- or 7-membered nitrogen heterocycle which optionally has a further heteroatom selected from the group consisting of O, S and N as ring member and which optionally carries 1, 2, 3 or 4 C₁-C₄-alkyl groups;
R¹⁶ is hydrogen, C₁-C₂-alkyl or C₁-C₄-alkoxy;
or an agriculturally acceptable salt of the compound I.

2. The compound of the formula I according to claim 1 in which X is halogen.

3. The compound of the formula I according to claim 1 in which A is a chemical bond.

4. The compound of the formula I according to claim 1 in which Y is a group O—R⁴ where R⁴ is as defined above.

5. The compound of the formula I according to claim 4 in which R⁴ is C₁-C₄-alkyl or C₃-C₄-alkenyl.

6. The compound of the formula I according to claim 1 in which R³ is hydrogen and R² is C₂-C₆-alkyl or trifluoromethyl or R¹ together with R² is C₂-C₆-alkylene.

7. The compound of the formula I according to claim 1 in which R² is a group (CH₂)ₖ—R^{b} in which k is 1 or 2 and R^{b} is as defined above.

8. The compound of the formula I according to claim 1 in which R³ is hydrogen, W is oxygen and Y is a group OR⁴ and in which the group of the formula 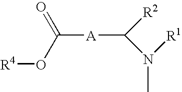
is derived from an α-amino acid or an ester thereof, where the α-amino acid is selected from the group consisting of proline, pipecolinic acid, leucine, isoleucine, methionine, phenylalanine, tyrosine and valine.

9. The compound of the formula I according to claim 1 in which m is 1, 2, 3 or 4 and L is selected from the group consisting of halogen, cyano, nitro, C₁-C₆-alkyl, C₁-C₆-haloalkyl, C₁-C₆-alkoxy and C₁-C₆-alkoxycarbonyl.

10. The compound of the formula I according to claim 9 in which the phenyl group substituted by Lₘ is a group of the formula 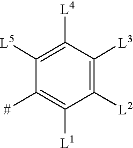
in which # is the point of attachment to the triazolopyrimidine skeleton and
L¹ is fluorine, chlorine, CH₃ or CF₃;
L², L⁴ independently of one another are hydrogen or fluorine;
L³ is hydrogen, fluorine, chlorine, cyano, CH₃, OCH₃ or COOCH₃; and
L⁵ is hydrogen, fluorine or CH₃.

11. The use of a compound of the formula I according to claim 1 for controlling phytopathogenic fungi.

12. A composition suitable for controlling harmful fungi, which composition comprises a solid or liquid carrier and a compound of the formula I according to claim 1.

13. A method for controlling phytopathogenic fungi, which method comprises treating the fungi or the materials, plants, the soil or seed to be protected against fungal attack with an effective amount of a compound of the formula I according to claim 1.

14. The compound of the formula I according to claim 2 in which A is a chemical bond.

15. The compound of the formula I according to claim 2 in which Y is a group O—R⁴ where R⁴ is as defined above.

16. The compound of the formula I according to claim 3 in which Y is a group O—R⁴ where R⁴ is as defined above.

17. The compound of the formula I according to claim 2 in which R³ is hydrogen and R² is C₂-C₆-alkyl or trifluoromethyl or R¹ together with R² is C₂-C₆-alkylene.

18. The compound of the formula I according to claim 3 in which R³ is hydrogen and R² is C₂-C₆-alkyl or trifluoromethyl or R¹ together with R² is C₂-C₆-alkylene.

19. The compound of the formula I according to claim 4 in which R³ is hydrogen and R² is C₂-C₆-alkyl or trifluoromethyl or R¹ together with R² is C₂-C₆-alkylene.

20. The compound of the formula I according to claim 5 in which R³ is hydrogen and R² is C₂-C₆-alkyl or trifluoromethyl or R¹ together with R² is C₂-C₆-alkylene.
